(19) United States
(12) Patent Application Publication
(10) Pub. No.: US 2015/0272588 A1
(43) Pub. Date: Oct. 1, 2015

(54) ENDOSCOPIC MULTI-CLIP DISPENSING APPARATUS

(71) Applicant: Mubashir H. Khan, Springfield, MO (US)

(72) Inventor: Mubashir H. Khan, Springfield, MO (US)

(21) Appl. No.: 14/276,513

(22) Filed: May 13, 2014

Related U.S. Application Data
(60) Provisional application No. 61/961,842, filed on Oct. 24, 2013.
(60) Provisional application No. 61/855,313, filed on May 14, 2013.
(60) Provisional application No. 61/957,306, filed on Jun. 29, 2013.

(51) Int. Cl. A61B 17/128; A61B 17/10; A61B 17/08

(52) U.S. Cl. CPC A61B 17/1285; A61B 17/083; A61B 17/105; A61B 2017/00336

(57) ABSTRACT

An endoscopic multi-clip dispensing apparatus has elongated inner and outer conduits sleeved together, wherein the inner conduit reciprocates between extension and retraction. A plunger rod pushes a line of clips through the core of the inner conduit at an indexed rate of one clip at a time. Each C-shaped clip has a pair of elongated jaws mated together at a hinge end. The clips are loaded into the inner conduit in head to tail fashion, with the jaw ends of a trailing clip pushing the hinge end of a leading clip. There further is a pair of wedge-shaped flared structures proximate the dispensing end of the conduits, for prying open the jaws of the lead clip during the progression of it being dispensed. Ultimately, after the lead clip passes clear of the flared structures, its jaws are free to snap shut and ‘bite’ into a target anatomy.

### CROSS-REFERENCE TO PROVISIONAL APPLICATION(S)

This application claims the benefit of U.S. Provisional Application No. 61/961,842, filed Oct. 24, 2013; U.S. Provisional Application No. 61/957,306, accorded filing date of Jun. 29, 2013; and, U.S. Provisional Application No. 61/855,313, accorded filing date of May 14, 2013. The foregoing disclosures are incorporated herein by this reference thereto.

### BACKGROUND AND SUMMARY OF THE INVENTION

The invention relates to surgery and, more particularly, to endoscopic multi-clip dispensing apparatus.

It is an object of the invention to line up a series of surgery clips in accordance with the invention in a line where the head of each clip is pushing into the tail of each preceding clip, until the leadmost clip.

It is another object of the invention to provide a dispensing apparatus in accordance with the invention for dispensing, at first, the leadmost clip, and then serially each succeeding clip thereafter when and where chosen by the operator (eg., surgeon).

A number of additional features and objects will be apparent in connection with the following discussion of the preferred embodiments and examples with reference to the drawings.

### BRIEF DESCRIPTION OF THE DRAWINGS

There are shown in the drawings certain exemplary embodiments of the invention as presently preferred. It should be understood that the invention is not limited to the embodiments disclosed as examples, and is capable of variation within the scope of the skills of a person having ordinary skill in the art to which the invention pertains. In the drawings,

FIG. 1 is a perspective view of a first embodiment of endoscopic multi-clip dispensing apparatus in accordance with the invention for dispensing a first embodiment of C-shaped surgery clips in accordance with the invention;

FIG. 2 is an enlarged scale perspective view of one such surgery clip, as with its jaws forced open;

FIG. 3 is a perspective view comparable to FIG. 2 except showing the clip in the state of normally snapped shut;

FIG. 3A is a sectional view taken along line III A-III A in FIG. 3;

FIG. 3B is a perspective view comparable to FIG. 3 except showing the composite construction of the clip wherein the spring portion is shown in solid lines (and is preferably a spring material like a spring steel) and the remaining portions shown in dashed lines might comprise a different material, such as non-spring stainless steel;

FIG. 4 is an enlarged scale perspective view of the dispensing end of said endoscopic multi-clip dispensing apparatus;

FIG. 5 is a perspective view comparable to FIG. 4 except showing a lead clip being advanced through the dispensing end of endoscopic multi-clip dispensing apparatus and out through the very end margin of the dispensing end's tubewall, wherein wedge-shaped, jaw-spreading flared ends of a pair of actuator arms are also extending through a driven extension stroke to project out the end of the dispenser;

FIG. 6 is a perspective view comparable to FIG. 5 except showing further advance of the process of dispensing of the lead clip;

FIG. 7 is a perspective view comparable to FIG. 6 except showing the lead clip being pushed further forward such that the wedge-shaped flared ends of the actuator arms have spread open wide the jaws of the clips (note, this is accomplished by the left and right flanges riding over the left and right flared ends);

FIG. 8 is an enlarged scale sectional view taken along the line VIII-VIII from FIG. 4;

FIG. 9 is an enlarged scale end elevational view taken in the direction of arrows IX-IX from FIG. 8;

FIG. 10 is an enlarged scale sectional view taken along the line X-X from FIG. 8;

FIG. 11 is an enlarged scale sectional view taken along the line XI-XI from FIG. 8;

FIG. 12 is an enlarged scale sectional view taken along the line XII-XII from FIG. 8;

FIG. 13 is a side elevational view, partly in section, take along line XIII-XIII from FIG. 8;

FIG. 14 is a side elevational view, partly in section, comparable to FIG. 13 except showing the coordinated extension of one flared end of one of the pair of actuator arms and projection of the lead clip out the dispensing end;

FIG. 15 is a side elevational view, partly in section, comparable to FIG. 14 except showing the end of travel for the extension of the one flared end of the one of the pair of actuator arms, in contrast to, the continued travel of the lead clip out the dispensing end;

FIG. 16 is a side elevational view, partly in section, comparable to FIG. 15 except showing the continued travel of the lead clip out of the dispensing end relative to the now stationary one flared end of the one of the pair of actuating arms, wherein the lead clip's flanges have engaged the wedge-shaped flared end and have been wedged apart such the jaws of the lead clip are spread wide open;

FIG. 17 is a side elevational view, partly in section, comparable to FIG. 16 except showing not only the clearance of the lead clip out of the dispensing end but also the clearance of the flanges past the one flared end of the one of the pair of actuating arms, whereby the jaws of the clip automatically start to spring shut;

FIG. 18 is a side elevational view, partly in section, comparable to FIG. 13 except omitting the illustration of any clip; and

FIG. 19 is an enlarged scale sectional view taken along line XIX-XIX from FIG. 1 and showing the abutting end of the urging follower that pushes on the last clip of the indefinitely long series of the first embodiment clips in accordance with the invention that would be loaded into the first embodiment endoscopic multi-clip dispensing apparatus in accordance with the invention;

FIG. 20 is a perspective view of another embodiment of endoscopic multi-clip dispensing apparatus in accordance with the invention for dispensing another embodiment of C-shaped surgery clips also in accordance with the invention;

FIG. 21 is an enlarged scale perspective view of one such surgery clip, as with its jaws in the state of normally snapped shut;

FIG. 22 is a perspective view comparable to FIG. 21 except showing the clip with its jaws in the state of forced open;

FIG. 23 is a top plan view of FIG. 21;

FIG. 24 is a side elevational view of FIG. 23, wherein dashed lines at the tail end show that varying the thickness of the tail end varies the clamping power of said clip;

FIG. 25 is an enlarged scale perspective view of the dispensing end of the dispensing apparatus of FIG. 20 and showing aspects of the dispensing arms of said dispensing apparatus;

FIG. 26 is a sectional view taken along line XXVI-XXVI in FIG. 25, except with the clip(s) removed from the view;

FIG. 27 is a sectional view taken along line XXVII-XXVII in FIG. 26;

FIG. 28 is a sectional view comparable to FIG. 26, except with the lead clip entering the ejection station for the dispensing end of said dispensing apparatus in accordance with the invention;

FIG. 29 is an enlarged scale end elevational view taken in the direction of arrows XXIX-XXIX from FIG. 28 and to show better show the ovate-shaped, jaw-spreading flared-ends of the actuator arms of said dispensing apparatus in accordance with the invention;

FIG. 30 is an enlarged scale sectional view taken in the direction of the arrows XXX-XXX from FIG. 28;

FIG. 31 is an enlarged scale sectional view taken along the line XXXI-XXXI from FIG. 28;

FIG. 32 is an enlarged scale sectional view taken along line XXXII-XXXII in FIG. 35 and comparable to FIG. 31 except wherein the lead tack has been advanced down the hollow passage of the flexible conduit of said dispensing apparatus in accordance with the invention (ie., by means of for example and without limitation being pushed from behind), and wherein the jaws of the lead clip have opened slightly;

FIG. 33 is an enlarged scale sectional view taken along line XXXIII-XXXIII in FIG. 35 and comparable to FIG. 32 except showing the plunger rod which serves as the pressure-urging follower that follows the trailing clip of the series of clips loaded head to tail in the hollow passage of the flexible conduit of said dispensing apparatus in accordance with the invention, wherein said plunger rod advances the whole series of clips forward in unison by abutting and applying pressure against the tail end of the trailing clip (see, eg., FIG. 35);

FIG. 34 is a perspective view comparable to FIG. 25 except showing the ejection process of the lead clip advancing through the dispensing end of the conduit such that the lead clip has moved relatively forward and that the actuator arms have thrust relative forward such that the ovate-shaped, jaw-spreading flared-ends thereof extend outside the dispensing end of said dispensing apparatus in accordance with the invention;

FIG. 35 is a perspective view comparable to FIG. 34 except showing the ejection process of the lead clip advancing through the dispensing end of the conduit such that the lead tack has moved relatively forward and that the ovate-shaped, jaw-spreading flared-ends of the actuator arms have begun to urge the jaws of the clip relatively apart;

FIG. 36 is a sectional view partly comparable to FIG. 27 except showing the arms partly advanced forward and showing the relative position of the lead clip;

FIG. 37 is a sectional view partly comparable to FIG. 36 except showing further advance of the arms and lead clip;

FIG. 38 is a sectional view partly comparable to FIG. 37 except showing further advance lead clip only, wherein the arms advanced to their further extension point in FIG. 37, and here in FIG. 38 the flared ends of the arms are in the middle of being spread apart from a normal position of flexed inward toward each other (see, eg., FIG. 36 or 37);

FIG. 39 is a sectional view partly comparable to FIG. 38 except showing further advance of the lead clip wherein, again, the arms do not advance any further, but, the flared ends do spread apart ever so slightly more;

FIG. 40 is a sectional view partly comparable to FIG. 28 except corresponding to the disposition of things in FIG. 36;

FIG. 41 is a sectional view partly comparable to FIG. 40 except corresponding to the disposition of things in FIG. 37;

FIG. 42 is a sectional view partly comparable to FIG. 41 except corresponding to the disposition of things in FIG. 38;

FIG. 43 is a sectional view partly comparable to FIG. 42 except corresponding to the disposition of things in FIG. 39;

FIG. 44 is a sectional view partly comparable to FIG. 43 except showing further advance of the lead clip, and, the beginning of the forcing apart the jaws thereof; and

FIG. 45 is a sectional view partly comparable to FIG. 43 except showing still further advance of the lead clip, and, the maximum extent of the forcing apart the jaws of the clip, after which said lead clip has been ejected clear of said dispensing apparatus and is free to clamp (eg., ‘bite’) on the target tissue which the user (eg., surgeon) has presumably taken aim upon (target tissue not shown);

FIG. 46 is a perspective view of a further embodiment of endoscopic multi-clip dispensing apparatus in accordance with the invention for dispensing a further embodiment of endoscopic surgery clips also in accordance with the invention;

FIG. 47 is an enlarged scale perspective view of one such surgery clip, as with its jaws spread open in state of full extension about 180° open wide;

FIG. 48 is a perspective view comparable to FIG. 47 except showing the surgery clip with its jaws in the process of clamping shut, but still about 30° apart;

FIG. 49 is an exploded perspective view of the clip, showing that its two jaws are resiliently biased to a shut (eg., clamping) position therefor by a torsion spring;

FIG. 50 is a perspective view of a series of such clips loaded head to tail in the hollow passage of the flexible conduit of said further embodiment of said dispensing apparatus in accordance with the invention, wherein a plunger rod advances the whole series of clips forward in unison by abutting and applying pressure against the tail end of the trailing clip (see, eg., FIG. 35);

FIG. 51 is a side elevational view of FIG. 47;

FIG. 52 is a side elevational view of FIG. 48;

FIG. 53 is a top plan view of FIG. 51;

FIG. 54 is a bottom plan view of FIG. 51;

FIG. 55 is a side elevational view of the opposite side of FIG. 51;

FIG. 56 is a left end elevational view of FIG. 51;

FIG. 57 is a right end elevational view of FIG. 51;

FIG. 58 is a sectional view taken along line LVIII-LVIII in FIG. 51;

FIG. 59 is a sectional view taken along line LIX-LIX in FIG. 51;

FIG. 60 is an elevational view, partly in section, of the dispensing end of FIG. 46 and/or somewhat of FIG. 50;

FIG. 61 is a section view taken along line LXI-LXI in FIG. 60;

FIG. 62 is an elevational view, partly in section, and comparable to FIG. 60 except showing the advance of the lead clip out of the dispensing end of the conduit of said dispensing apparatus;

FIG. 63 is an elevational view, partly in section, and comparable to FIGS. 60 and 62 except showing the ejection of the lead clip out of the dispensing end of the conduit of said dispensing apparatus, wherein this view presupposes that the operator (eg., surgeon) of this dispensing apparatus has hooked the lead hook-end of the clip on some target anatomy (not shown), which is why the lead hook-end of the clip remains axially aligned with the core of the conduit of the dispensing apparatus and it is the trailing hook-end of the clip which swings around to ‘bite into’ (eg., form a clamp with) the lead hook-end;

FIG. 64 is an exploded perspective view comparable to FIG. 49, except of still another embodiment of an endoscopic surgery clip in accordance with the invention, which like the clip of FIG. 49 has two jaws which are torsionally-biased toward each other, and which can be spread 180° apart, except wherein the torsion applicator is bar of resilient material (which is shown suspended in isolation between the two jaws of the clip);

FIG. 65 is an enlarged-scale side elevational view comparable to FIG. 50, except of the clip of FIG. 64, and of more particularly the hinge portion thereof;

FIG. 66 is a sectional view taken along line LXVI-LXVI in FIG. 65, wherein the torsional resilient bar has been twisted into at least one full coil, whereby the uncoiling thereof into half a coil swings the jaws from wide open at 180° apart to shut at about 0° apart;

FIG. 67 is a perspective view comparable to FIG. 47, except of yet another embodiment of a clip in accordance with the invention for dispensing out of a multi-clip dispensing apparatus in accordance with the invention wherein the clips are loaded in a line in a core of an axially-elongated conduit of the dispensing apparatus, in a head to tail line up;

FIG. 68 is a side elevational view thereof, with a leading hook-end for a leading jaw to the left in the view and a trailing hook-end for a trailing jaw shown to the right in the view;

FIG. 69 is a side elevational view comparable to FIG. 68 except showing the jaws of the clip flexed open beyond a mere 180° apart, and, instead to nearly 225° apart, wherein this view presupposes that the leading jaw has cleared the dispensing end of the dispensing apparatus and the operator (eg., surgeon) of the dispensing apparatus has hooked the lead hook-end of the clip on some target anatomy (not shown), which is why the lead hook-end of the clip is bent upward of axis of the the core of the conduit of the dispensing apparatus (as the trailing hook-end, which is still confined therein, remains axially-aligned with such core of the conduit of the dispensing apparatus);

FIG. 70 is a side elevational view comparable to FIG. 69 except showing the progression of the trailing jaw after it has cleared the dispensing end of the dispensing apparatus, ie., the jaws snap shut and “bite” into (eg., apply a clamping force on) the target tissues aimed at by the operator of the dispensing apparatus (eg., a surgeon); around to ‘bite into’ (eg., form a clamp with) the lead hook-end;

FIG. 71 is a side elevational view comparable to FIG. 68, except including the illustration of the dispensing end of the elongated conduit of the dispensing apparatus;

FIG. 72 is a side elevational view comparable to FIG. 69, except including the illustration of the dispensing end of the elongated conduit of the dispensing apparatus;

FIG. 73 is a side elevational view comparable to FIG. 70, except including the illustration of the dispensing end of the elongated conduit of the dispensing apparatus; and

FIG. 74 is an enlarged-scale side elevational view comparable to FIG. 69, and focusing in on the hinge-portion of said clip;

FIG. 75 is a perspective view comparable to any of FIG. 7 or 35 except of a different embodiment of a clip and dispensing apparatus in accordance with the invention, wherein unlike FIGS. 1-45 in which the clips have just a pair of jaws, the clip in this view has three jaws (ie., it is more of a claw) and the dispensing apparatus is arranged with three arms formed with flared ends to manage the opening of the jaws/claws of this clip, however, like the clips of FIGS. 1-45 the clips of this view does travel through the inner tubular conduit of the dispensing apparatus with the the jaws/claws facing forwardly; and

FIG. 76 is a section view taken along line LXXVI-LXXVI in FIG. 75.

### DETAILED DESCRIPTION OF THE PREFERRED EMBODIMENTS

The drawing figures show various embodiments of a series of surgery clips in accordance with the invention which are formed in a line in an tubular conduit of endoscopic multi-clip dispensing apparatus in accordance with the invention.

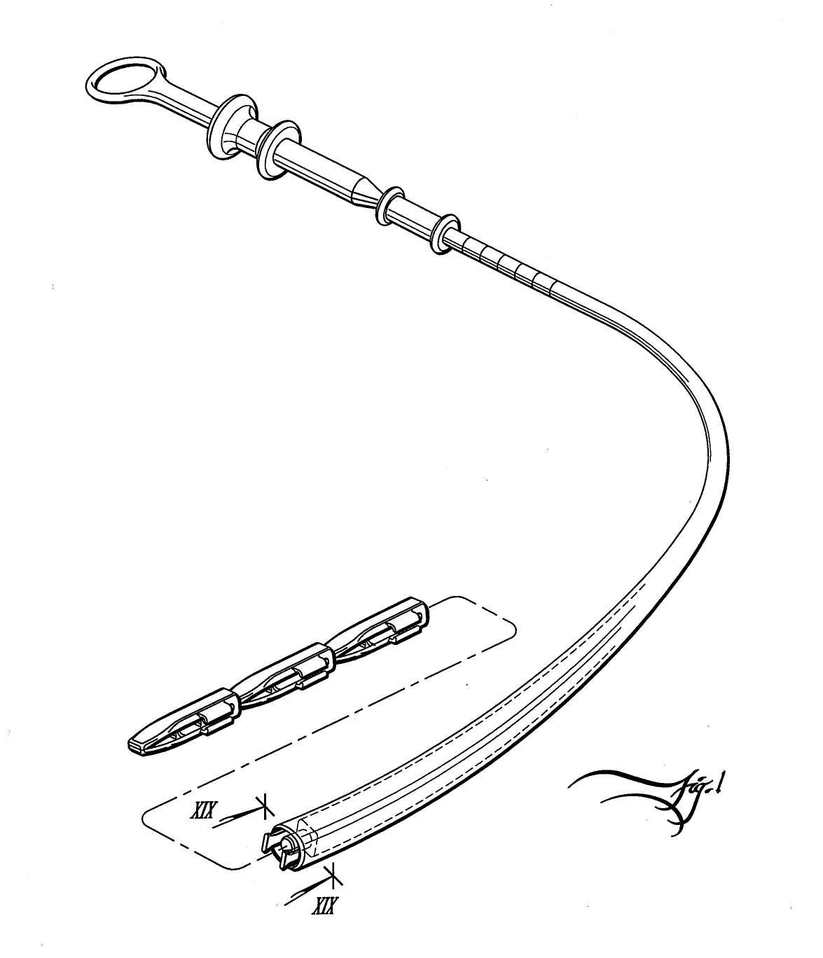

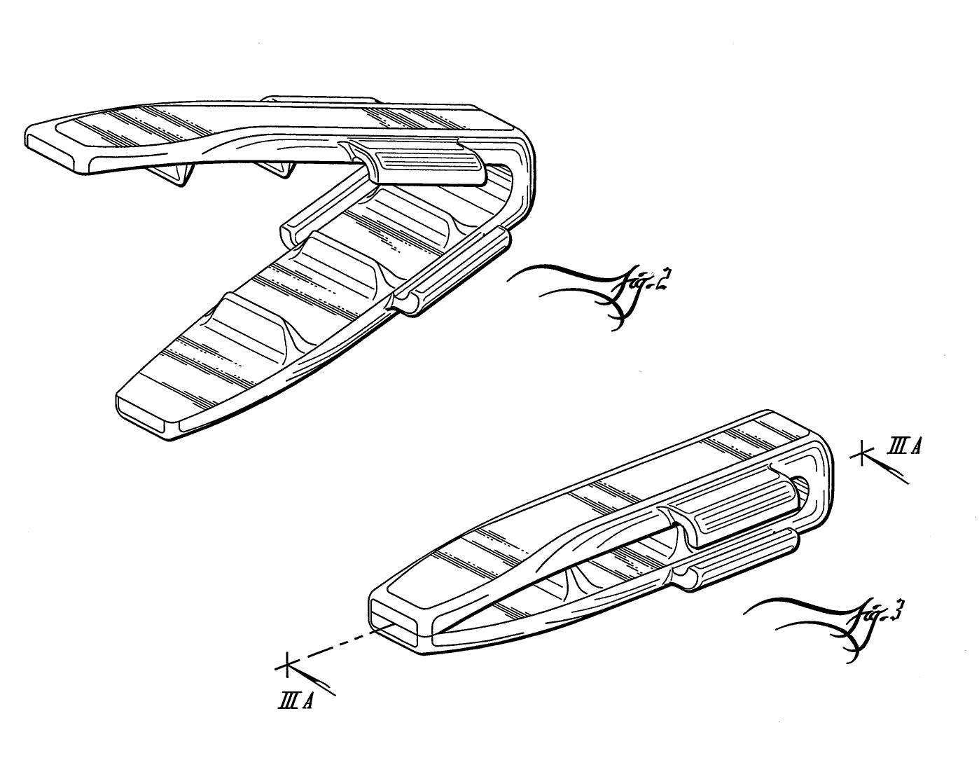

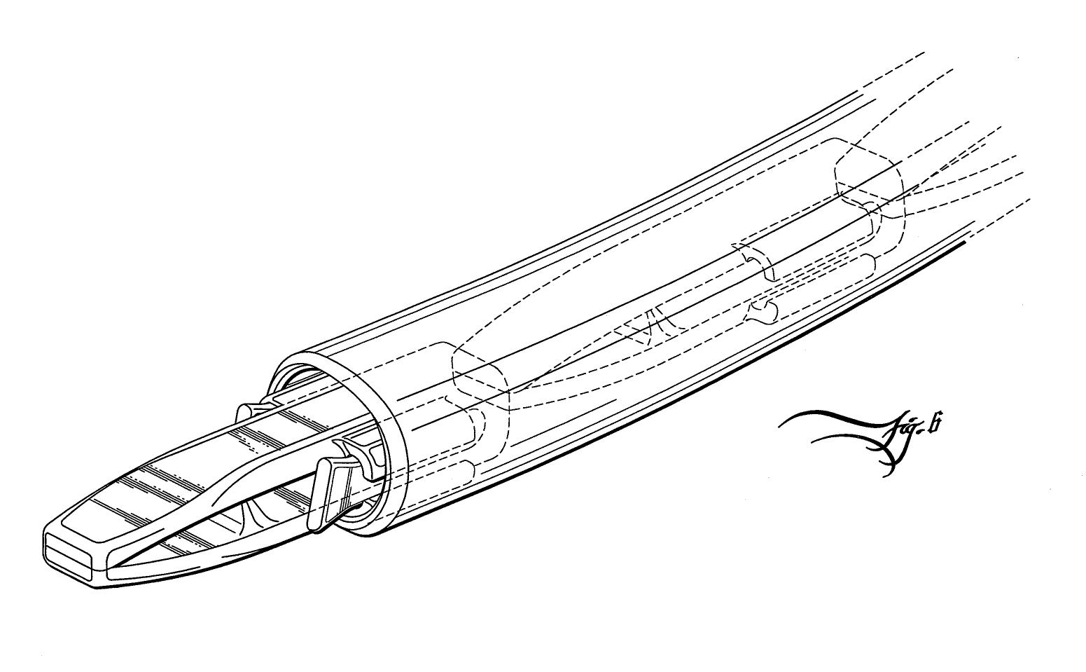

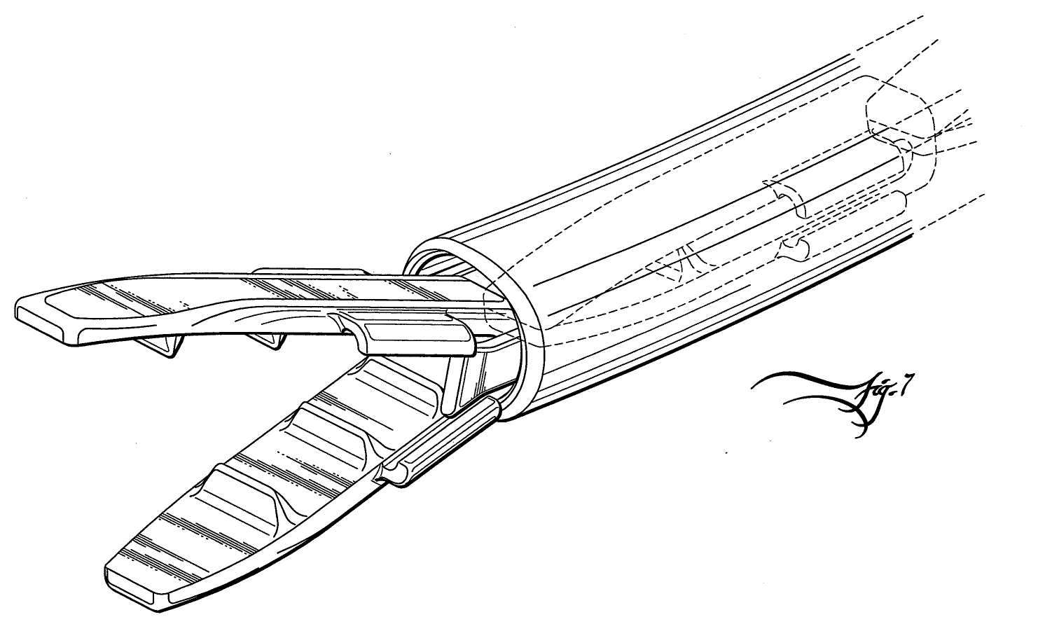

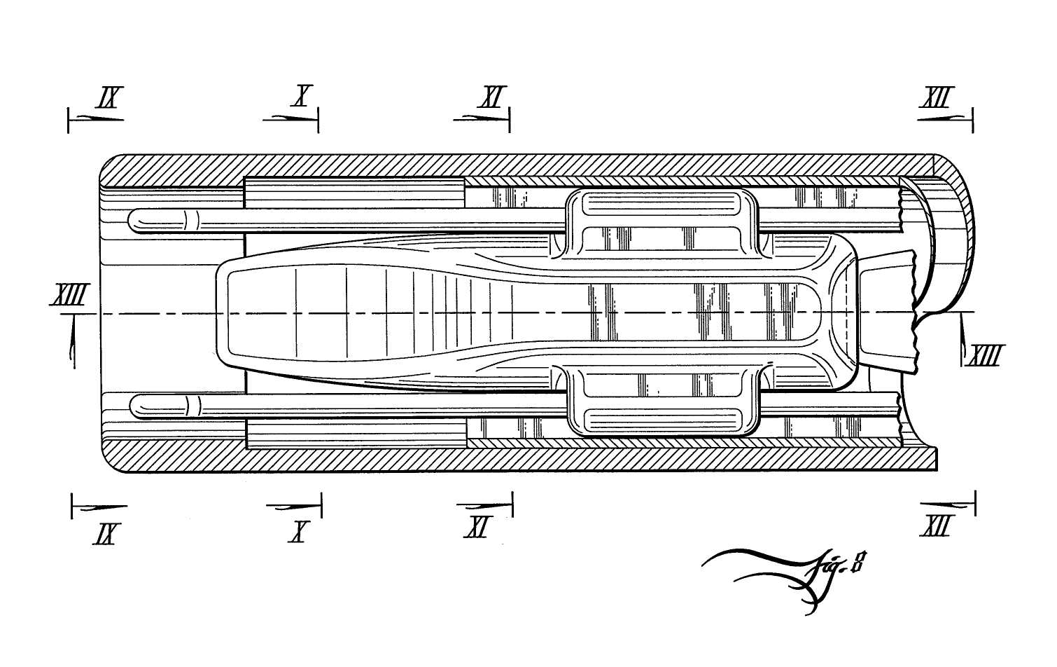

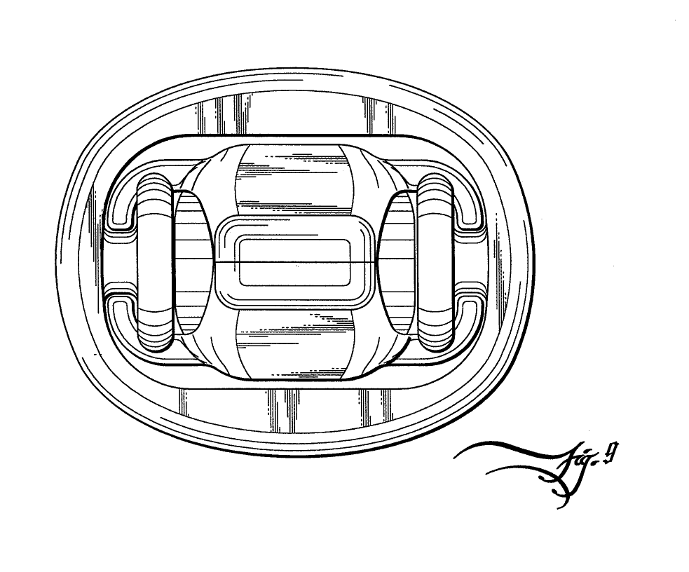

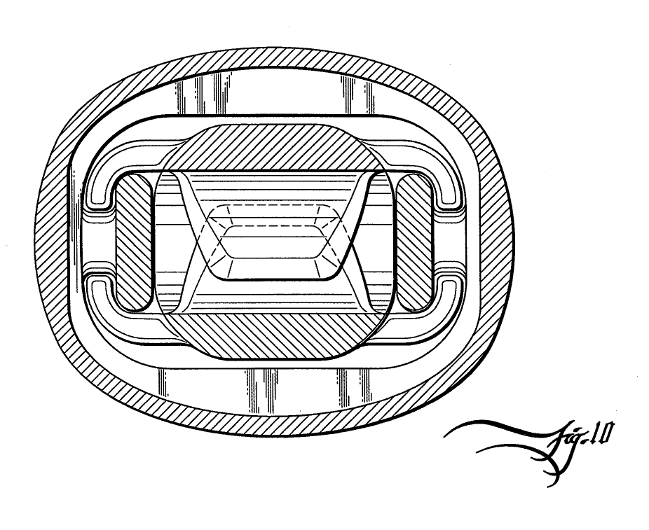

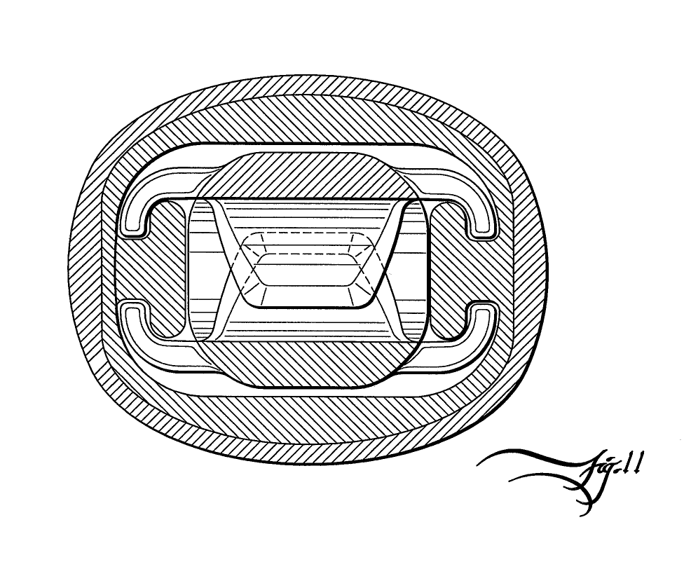

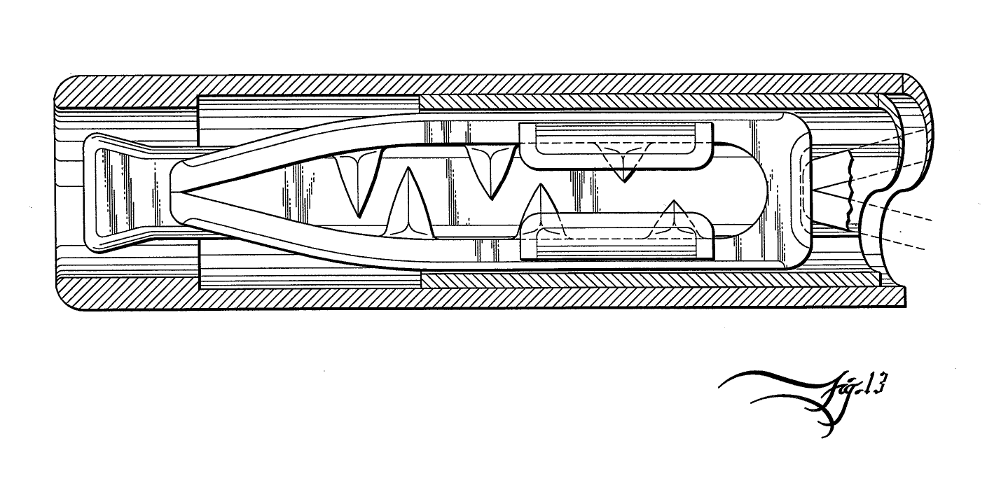

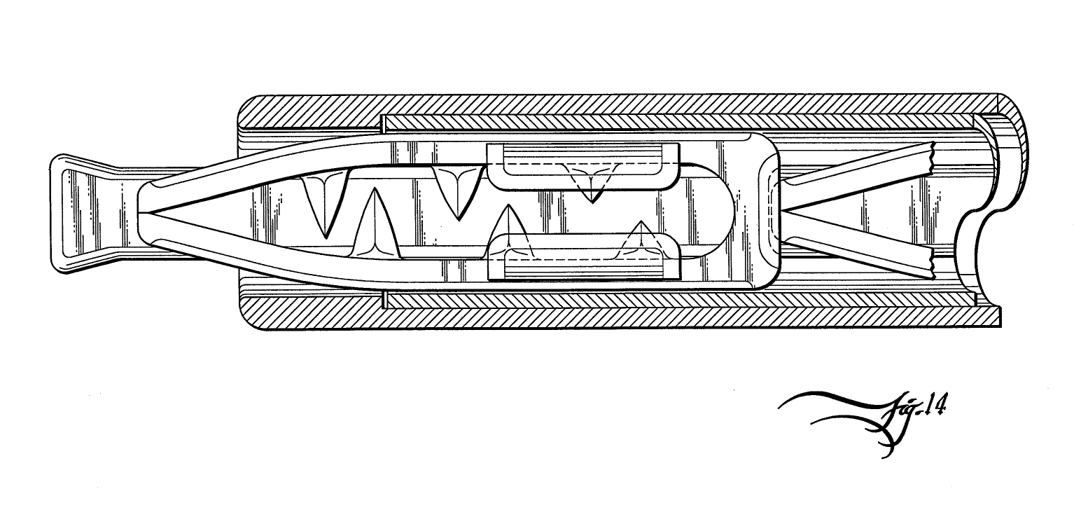

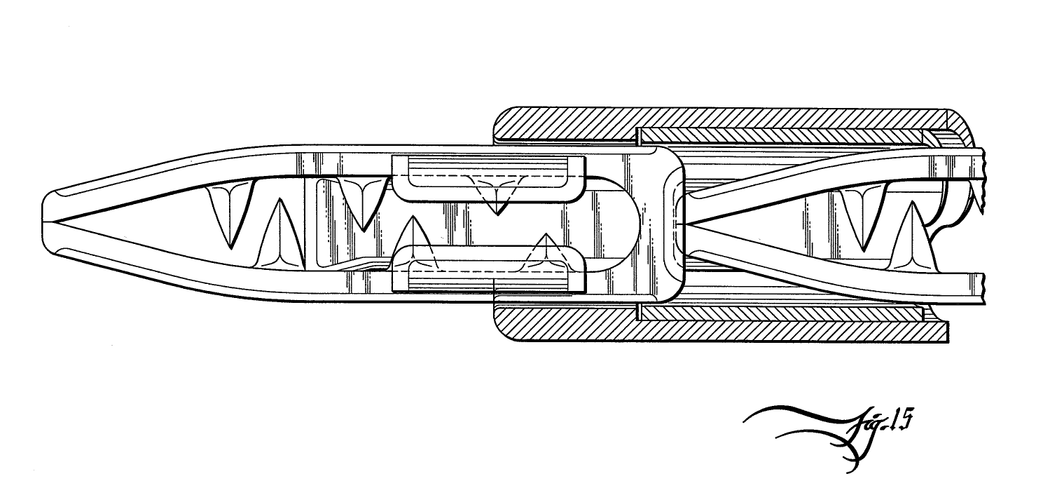

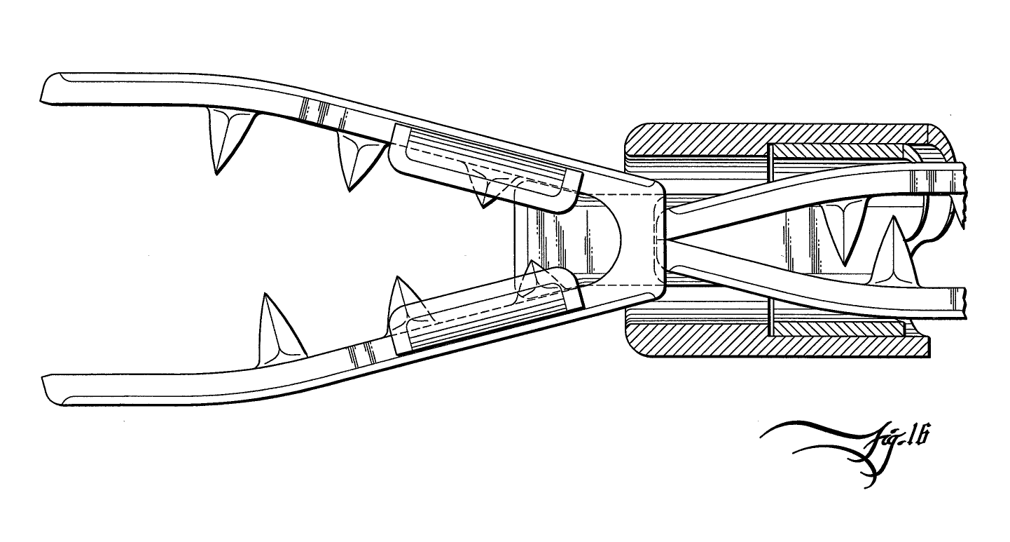

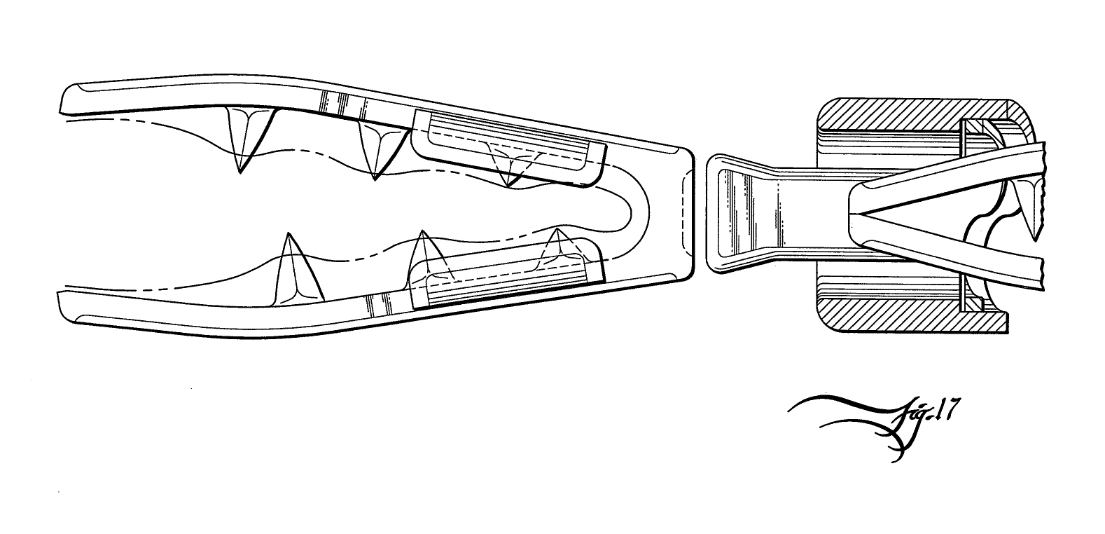

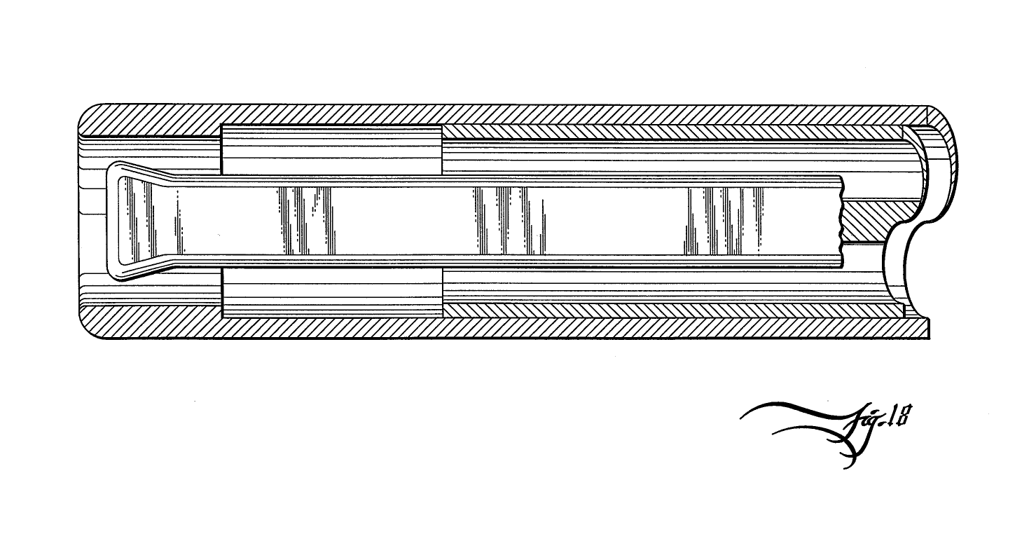

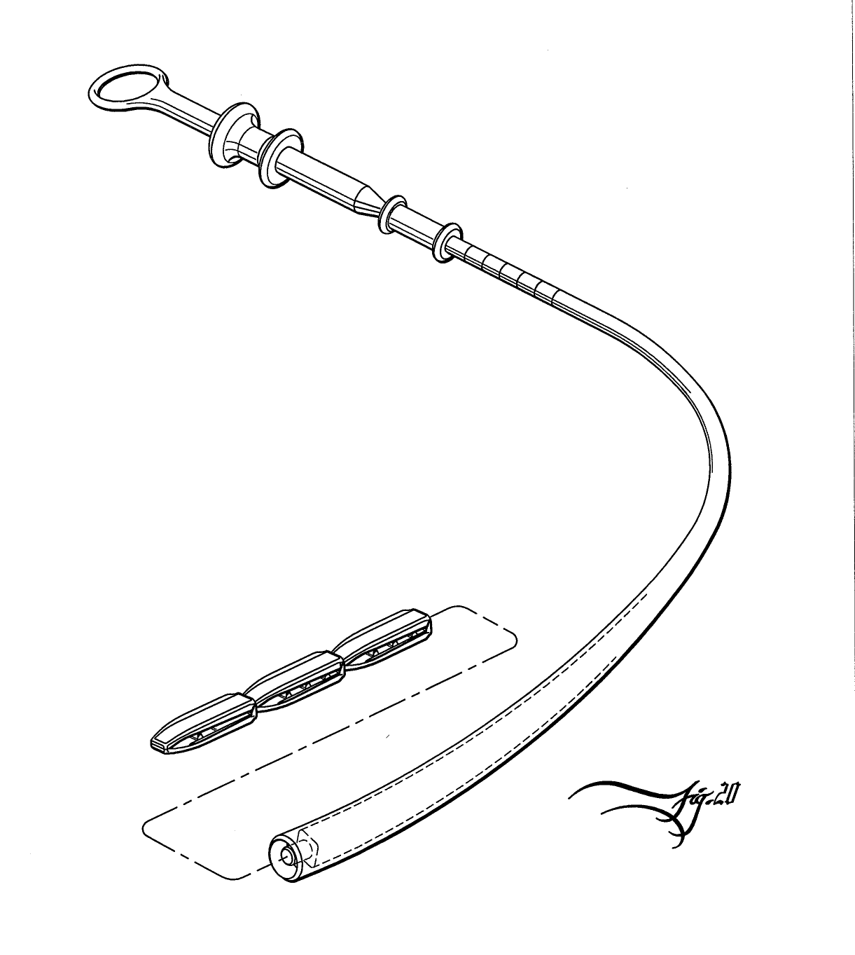

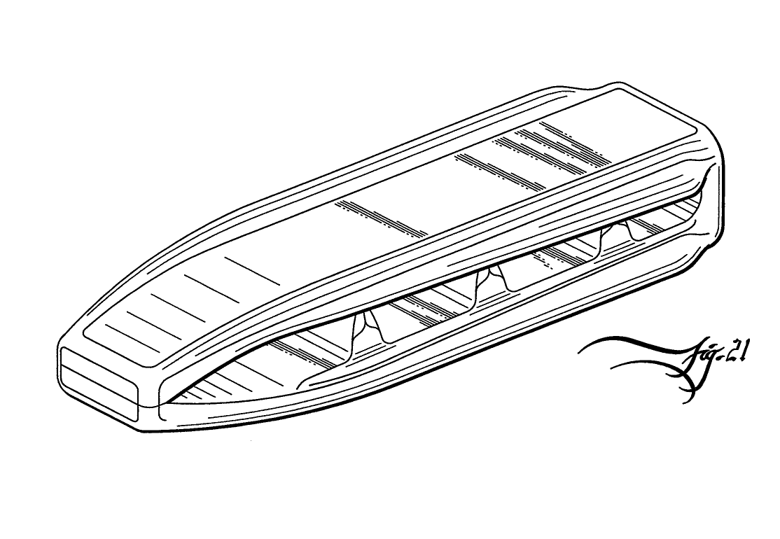

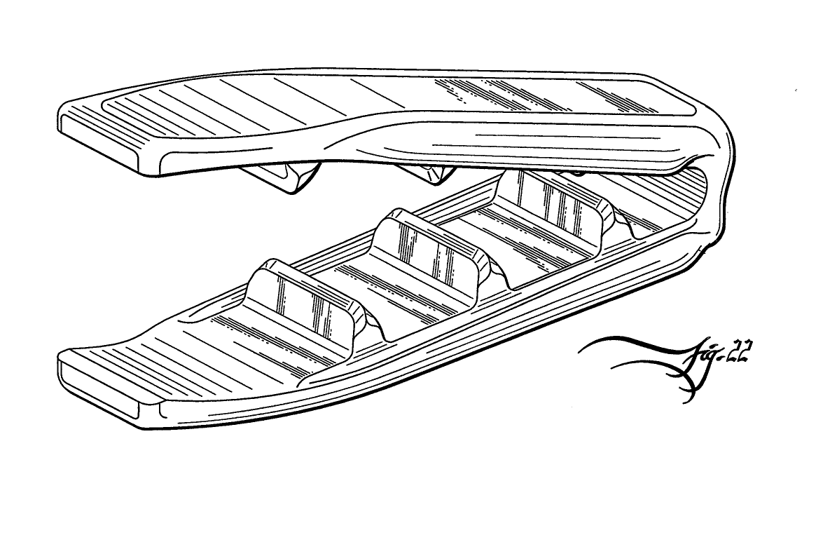

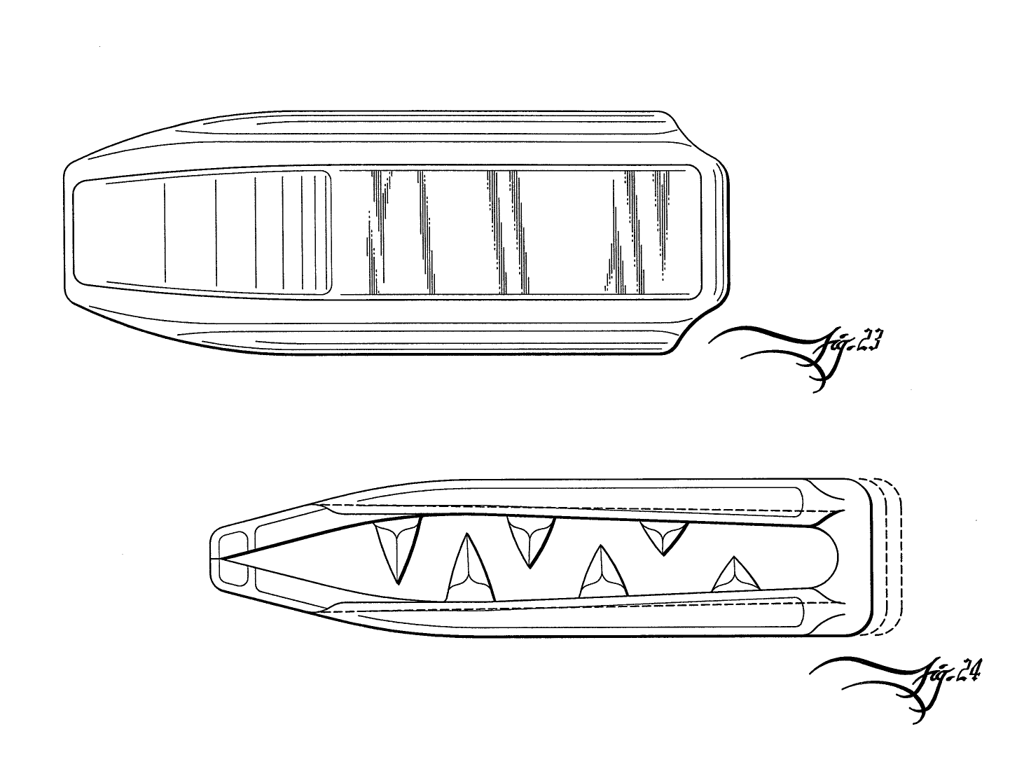

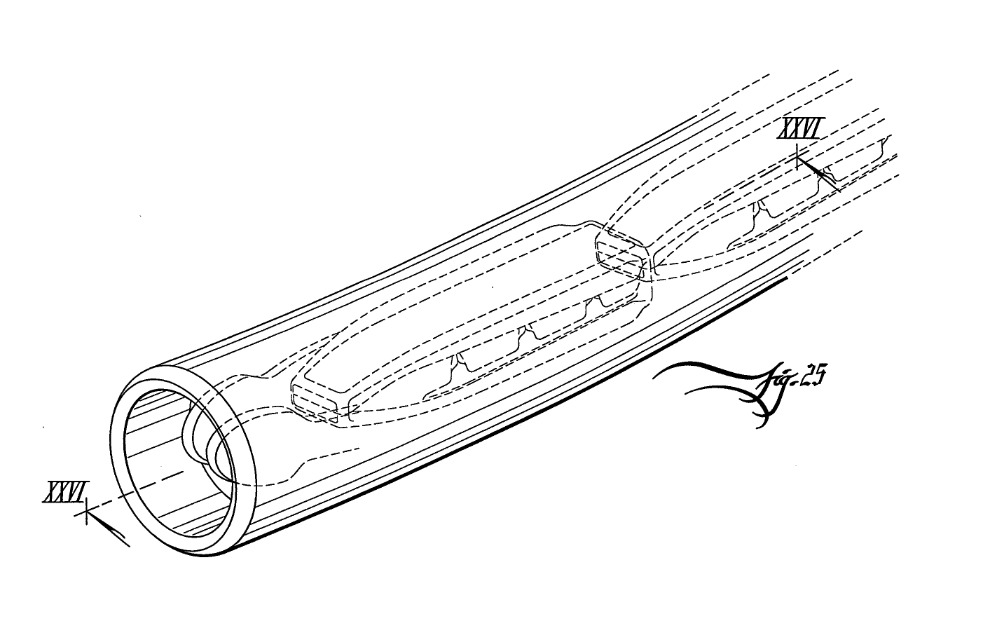

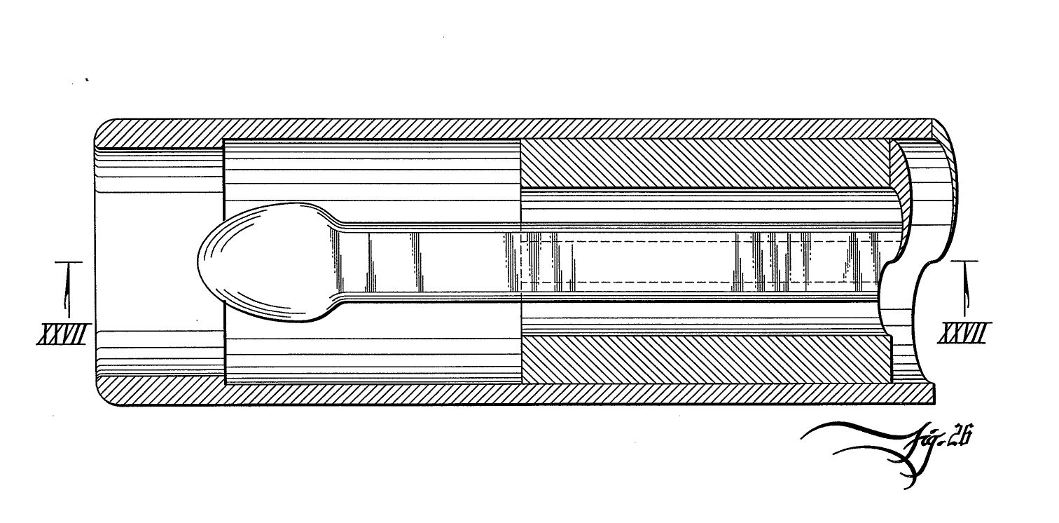

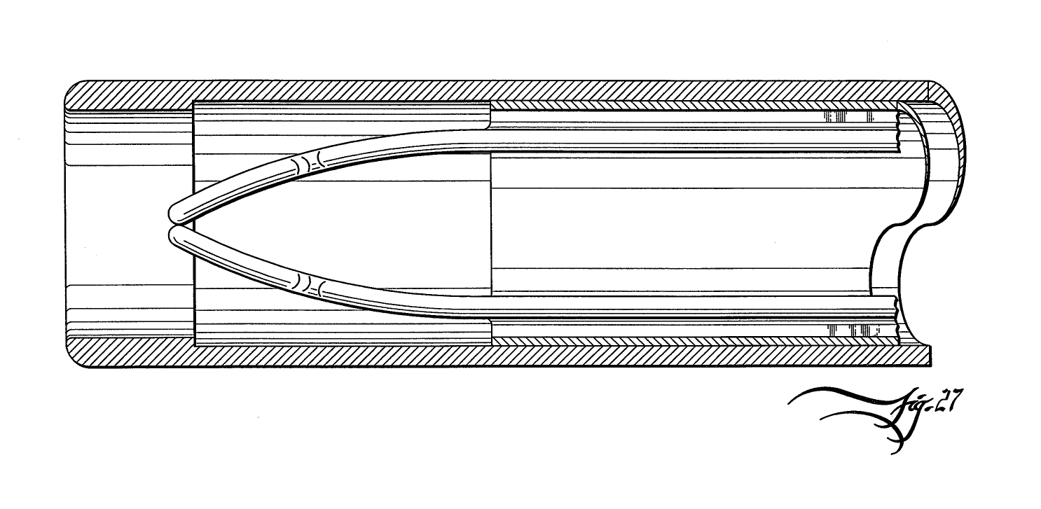

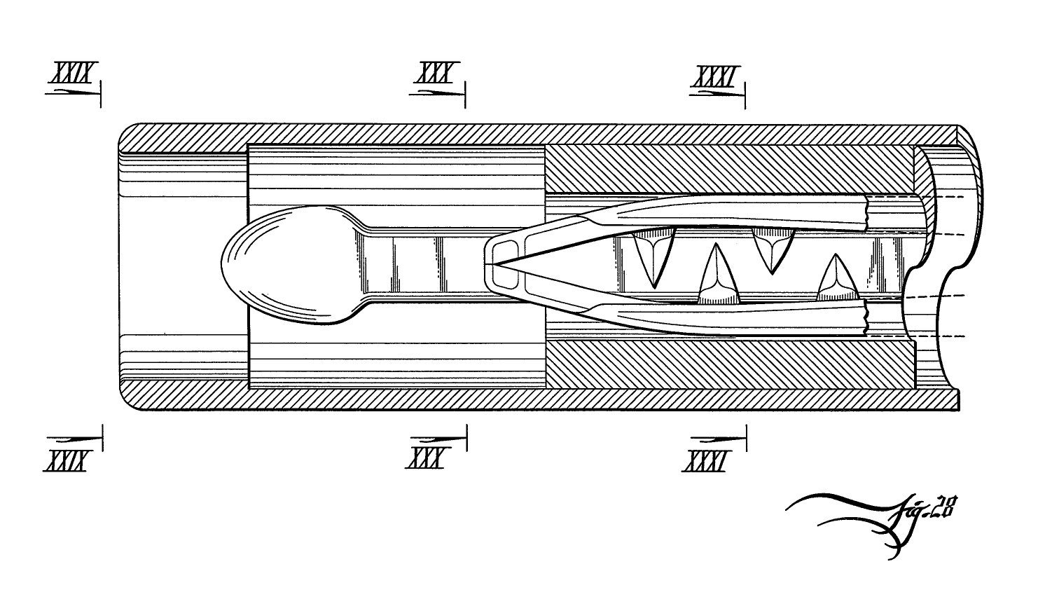

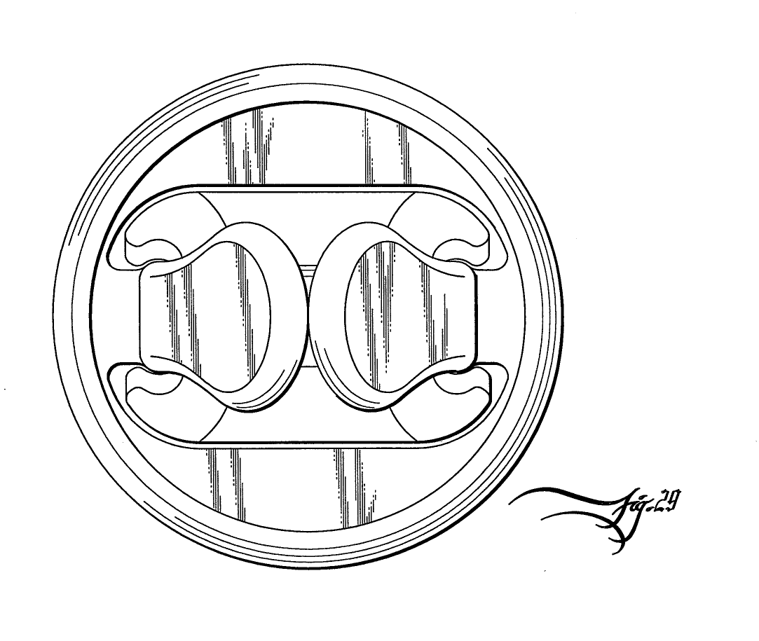

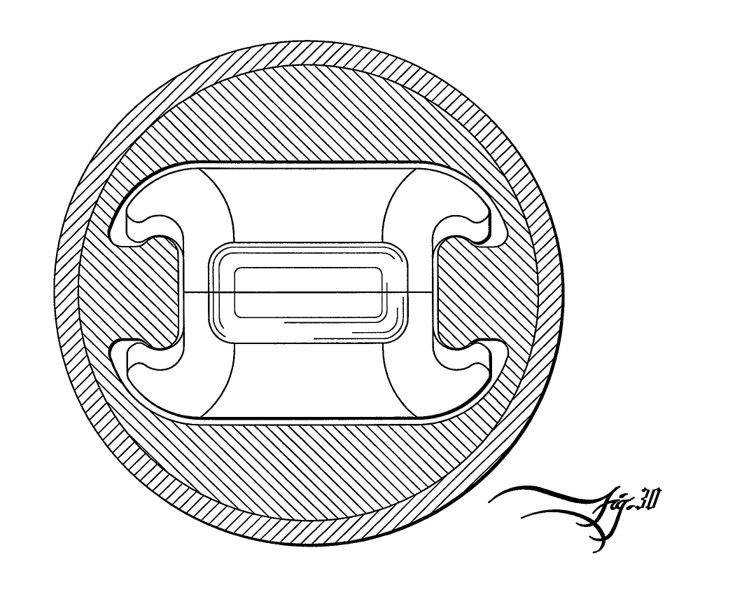

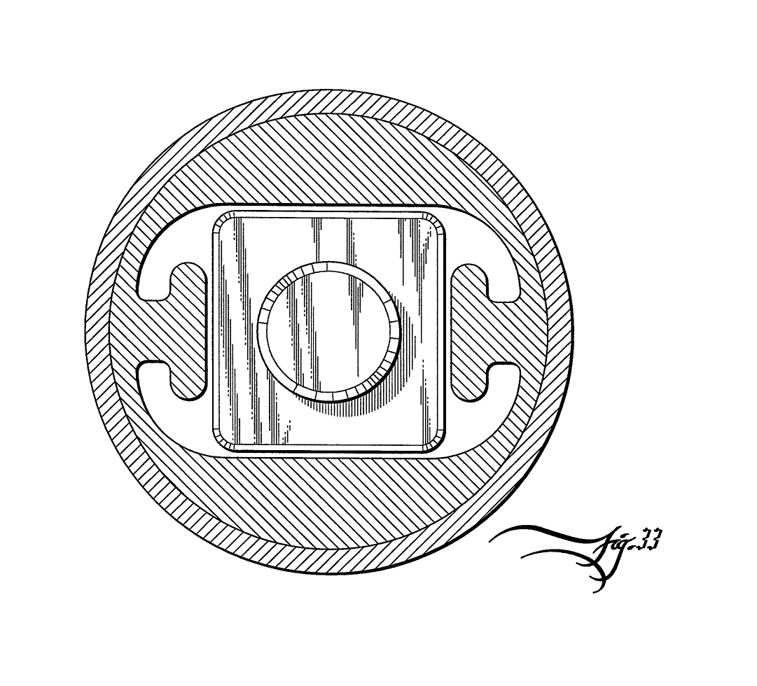

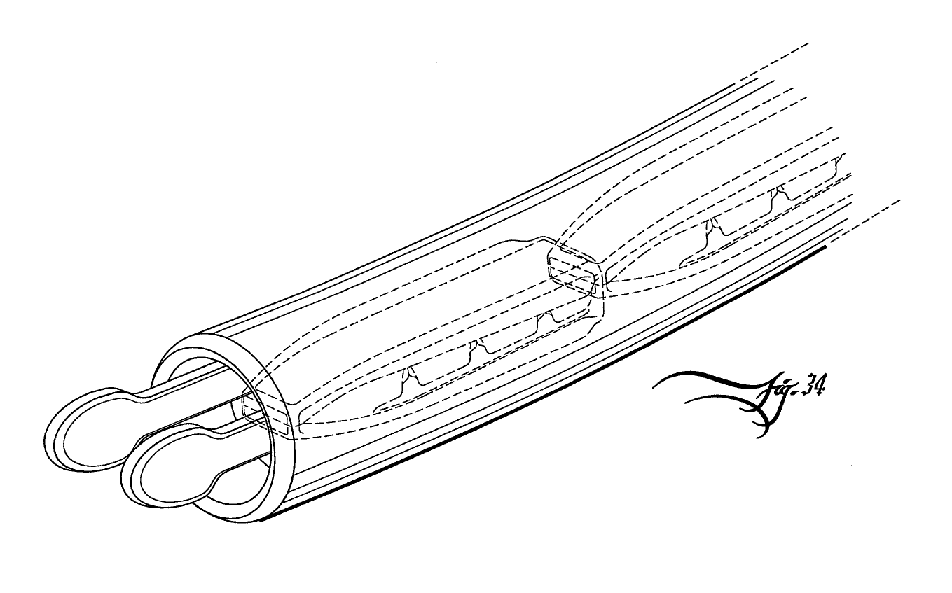

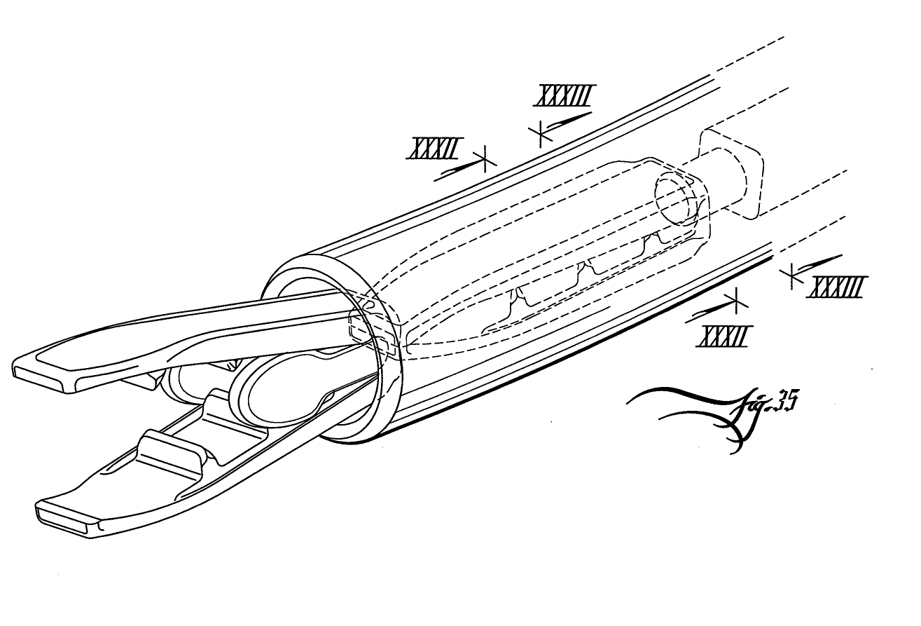

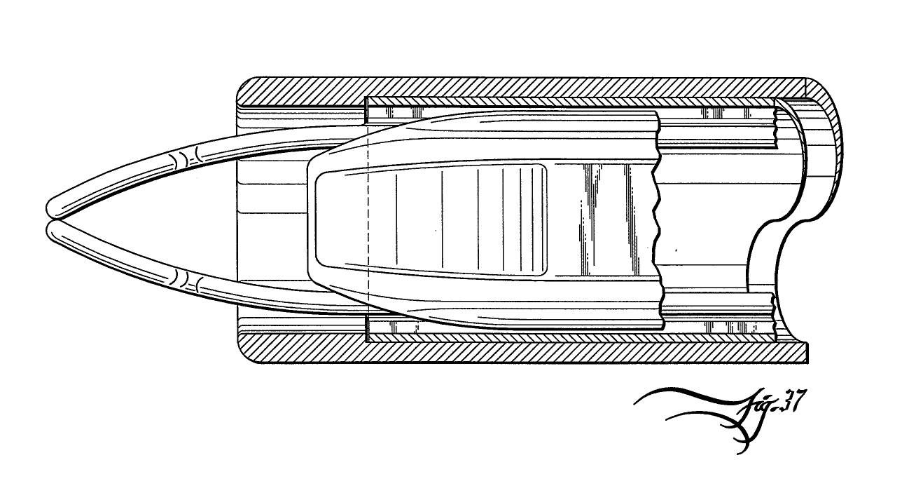

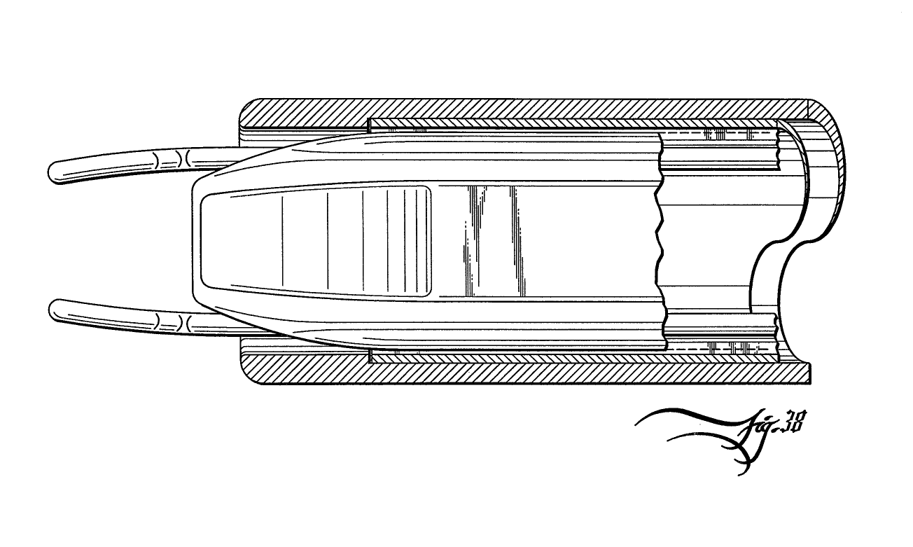

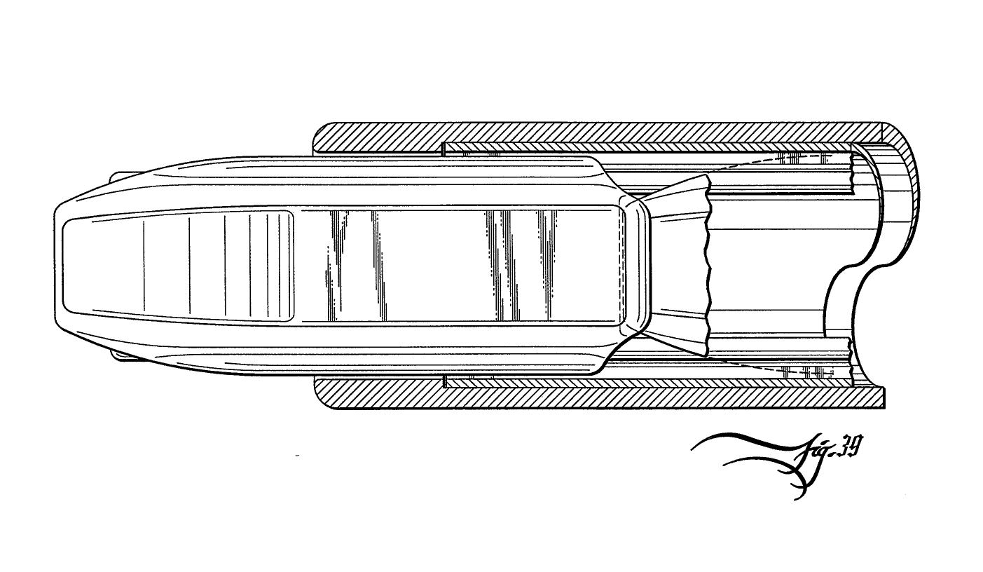

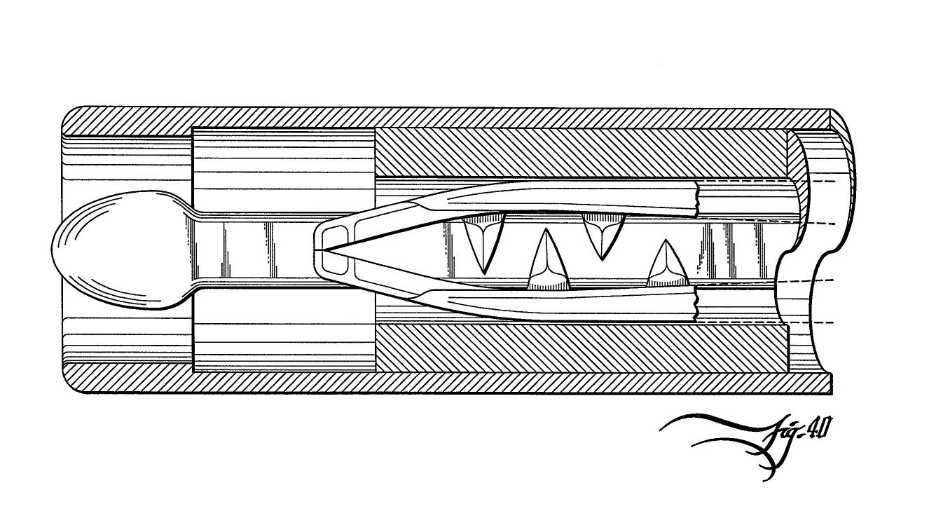

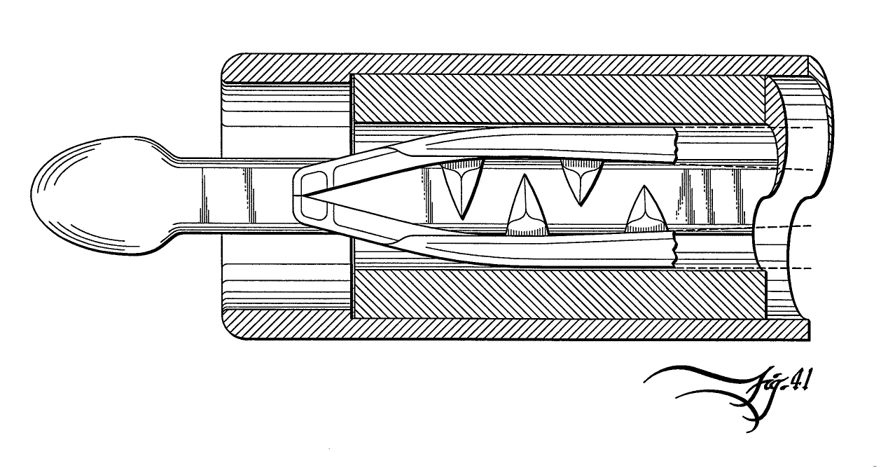

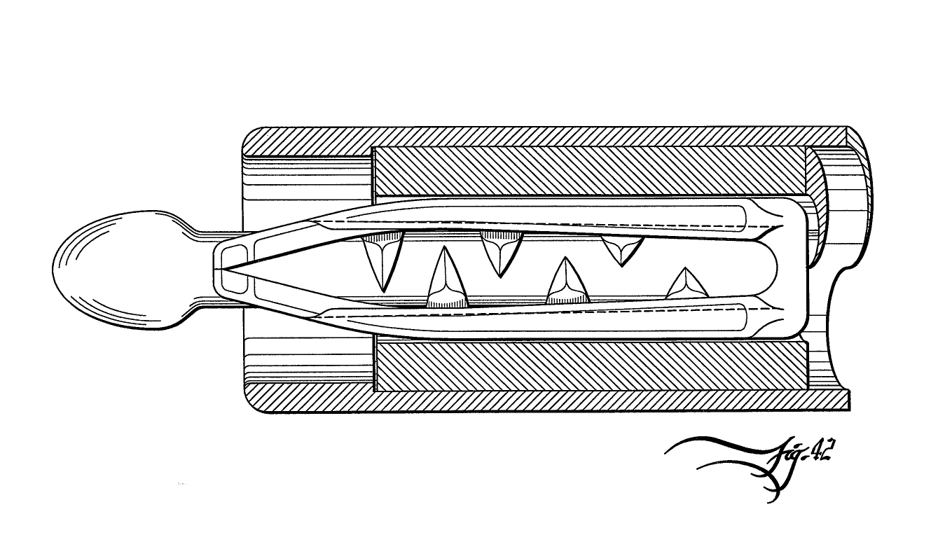

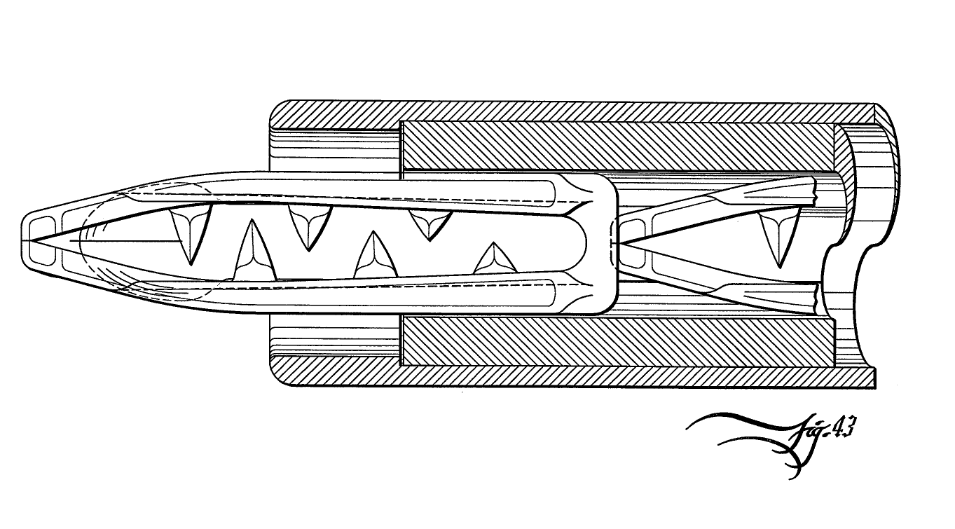

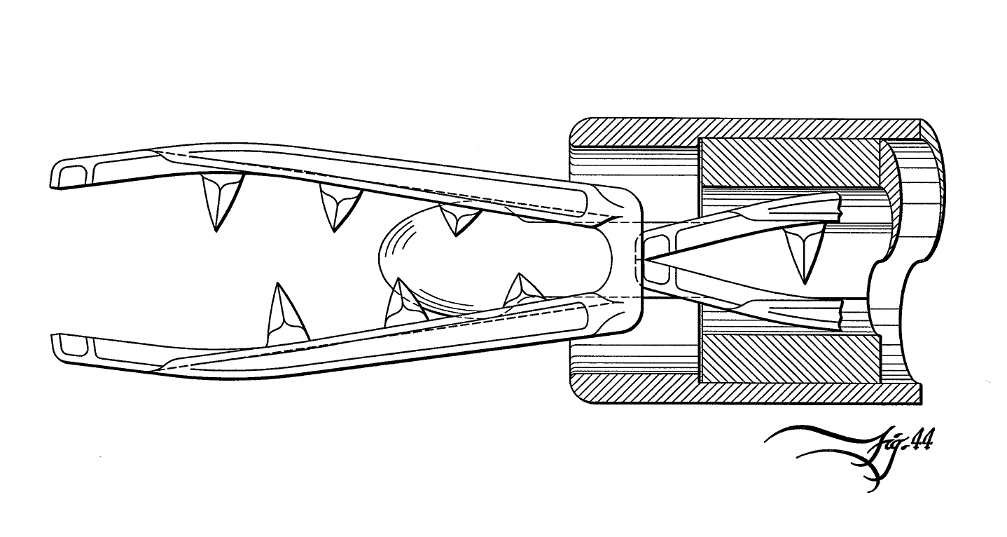

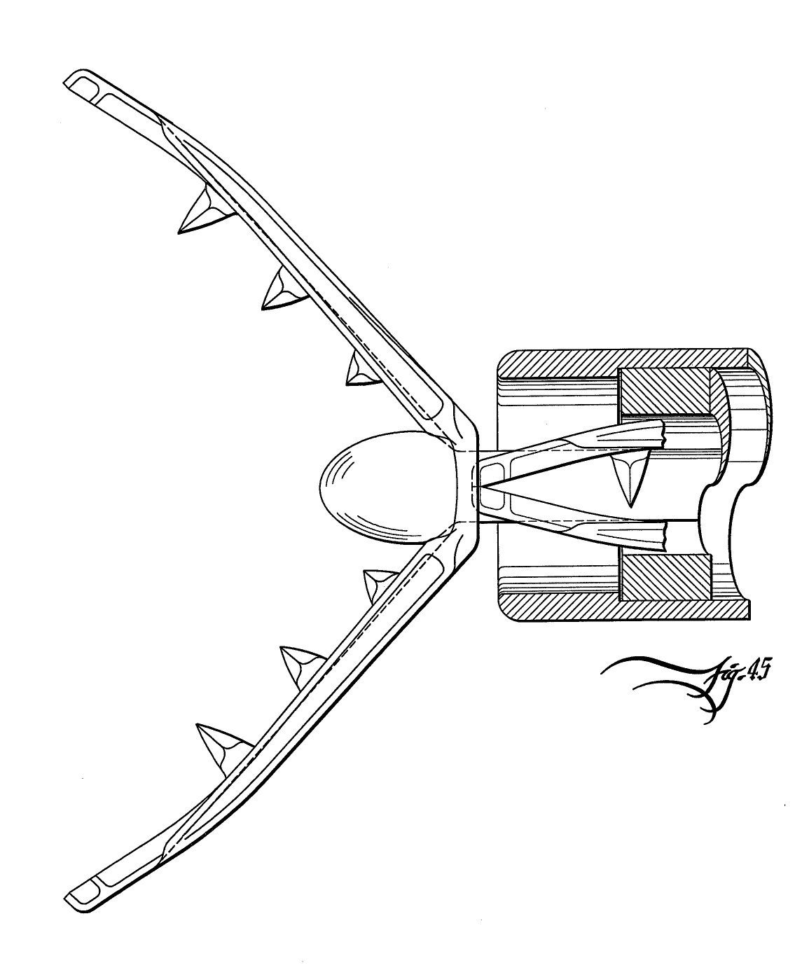

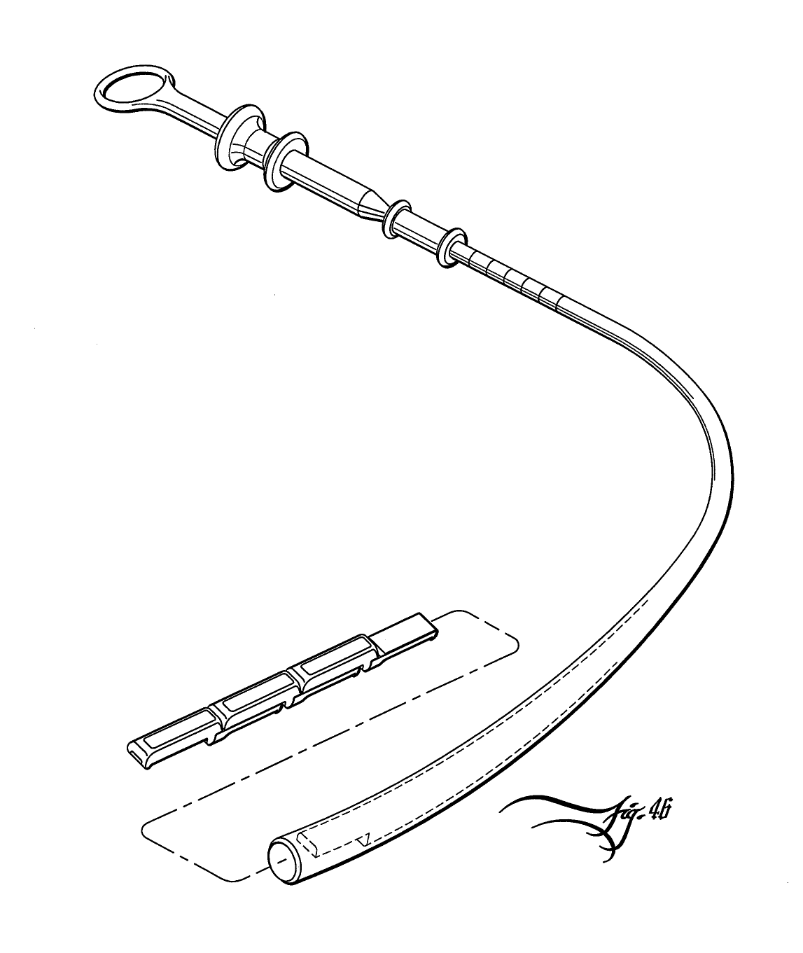

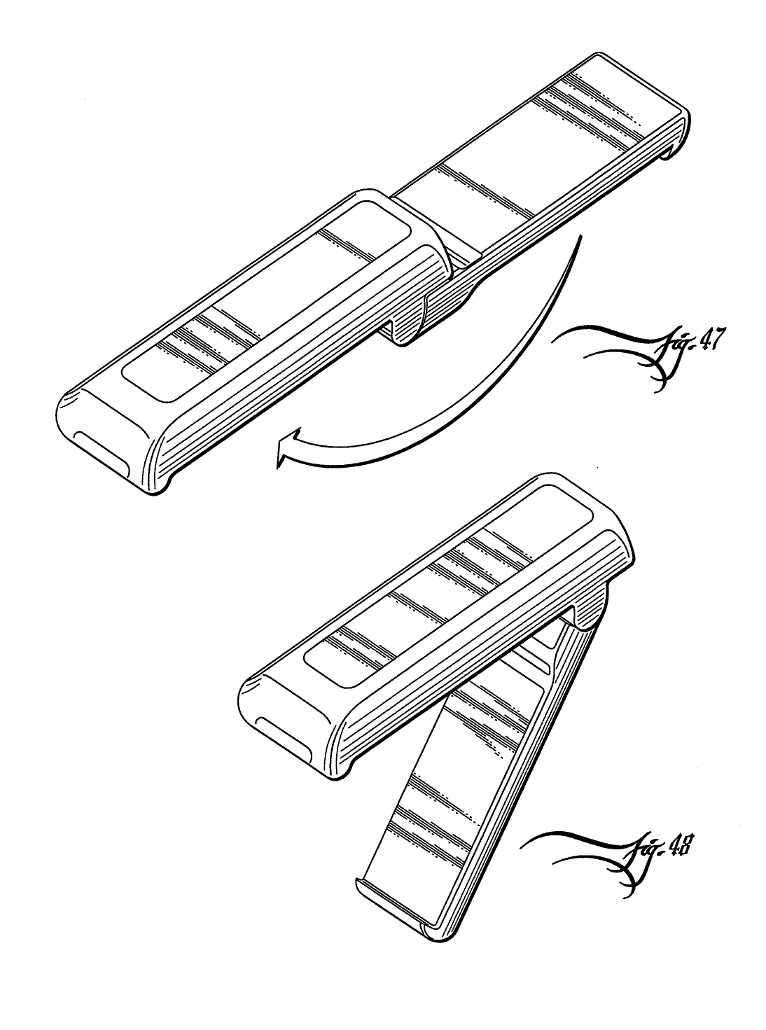

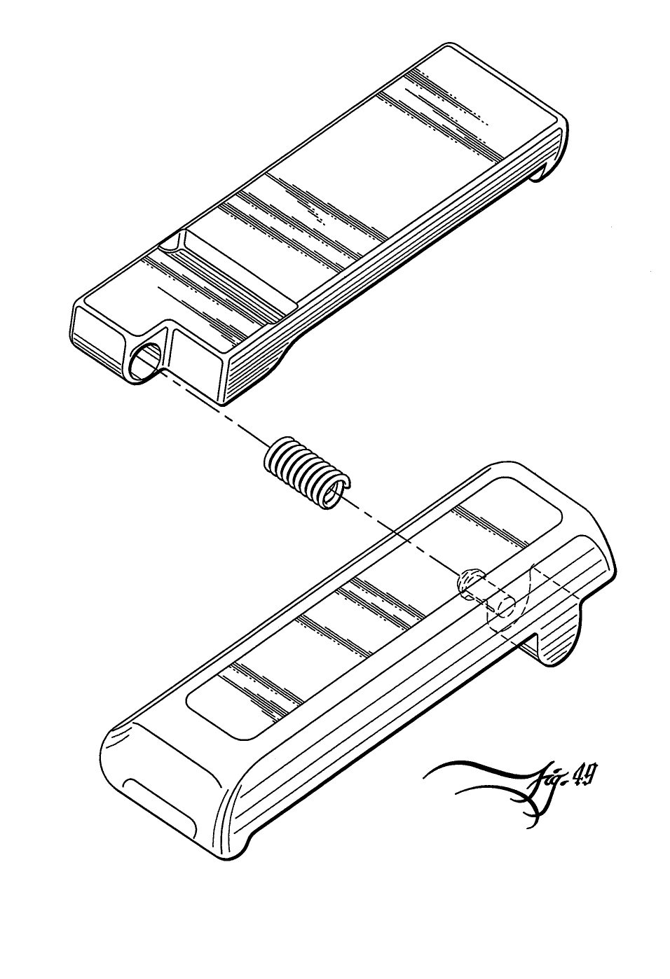

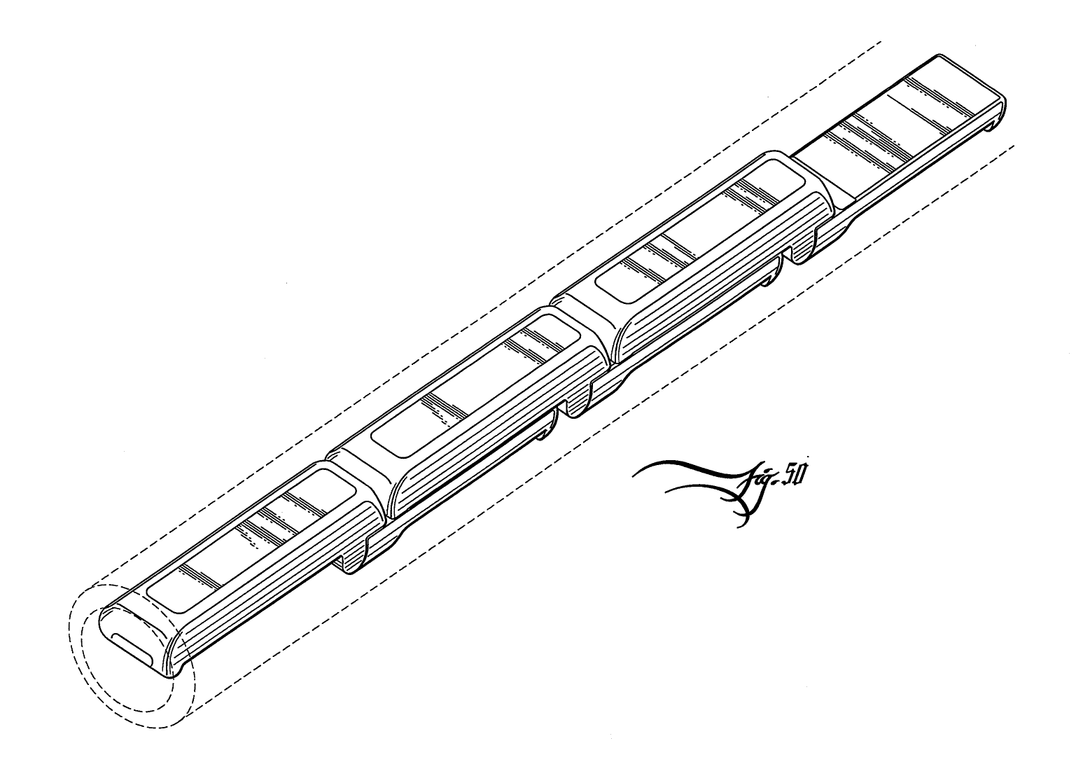

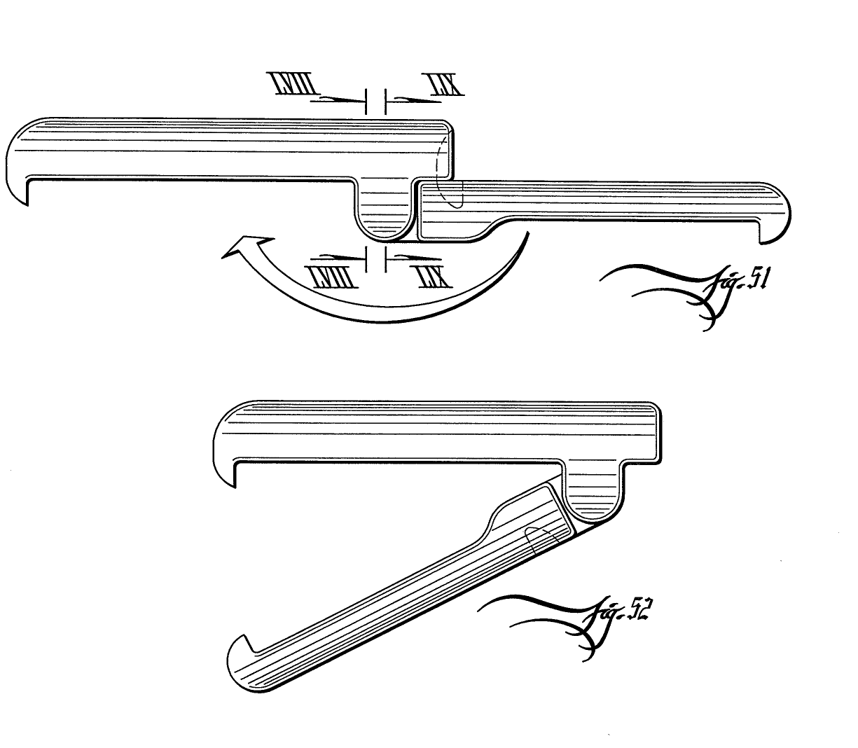

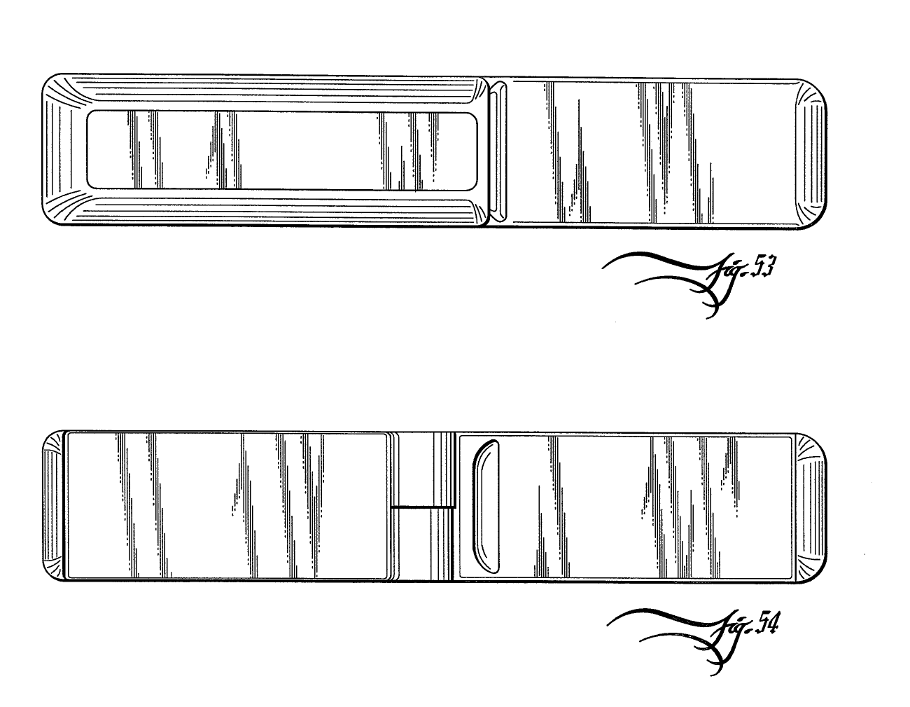

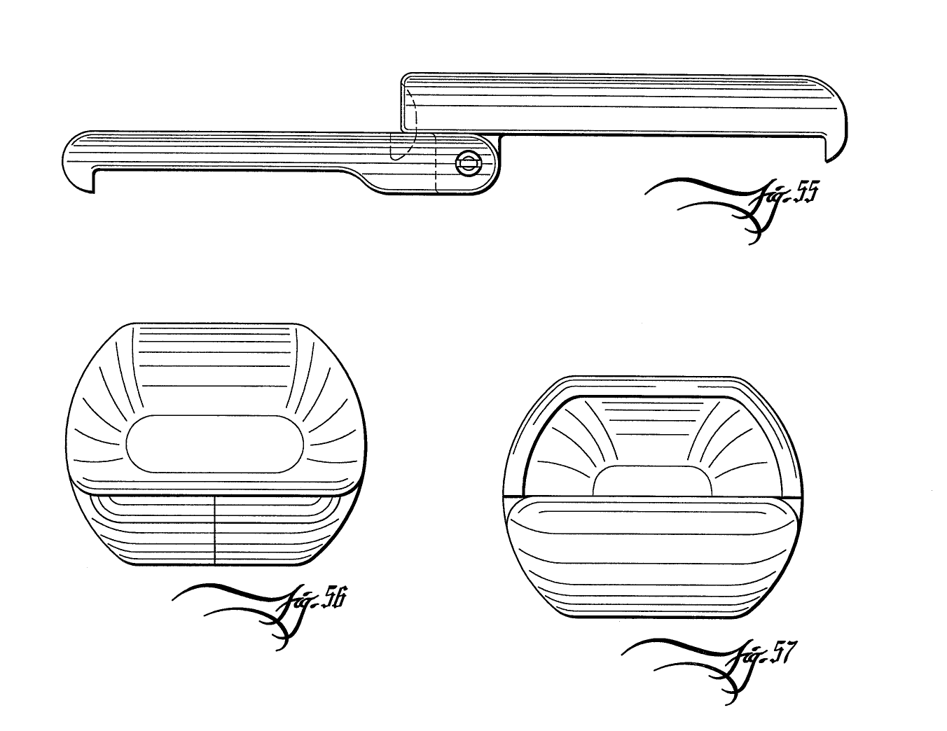

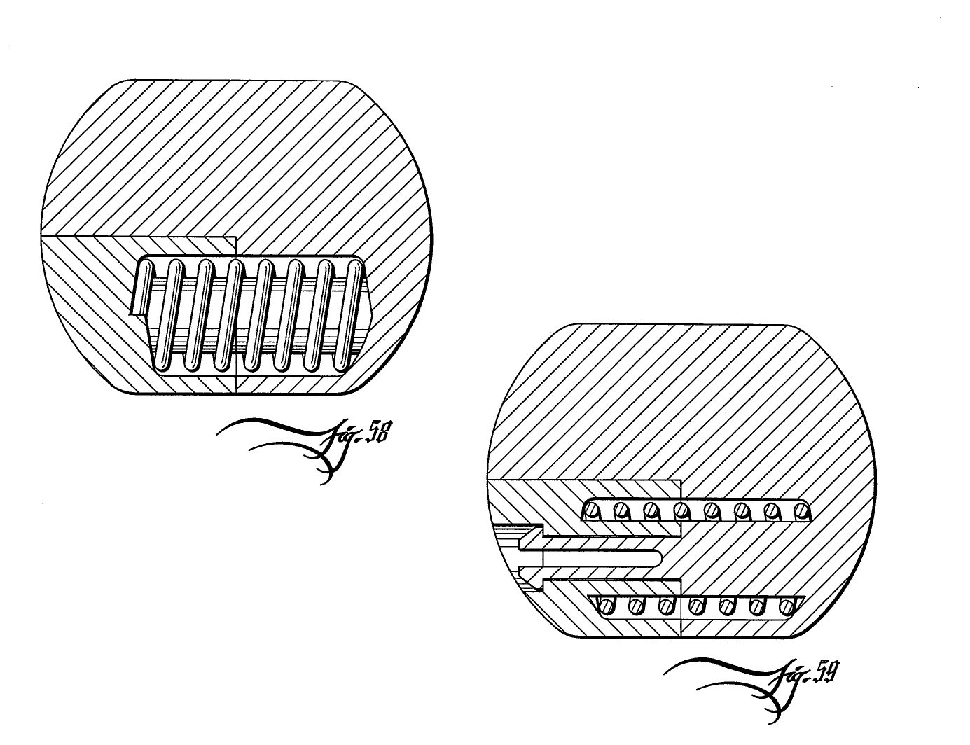

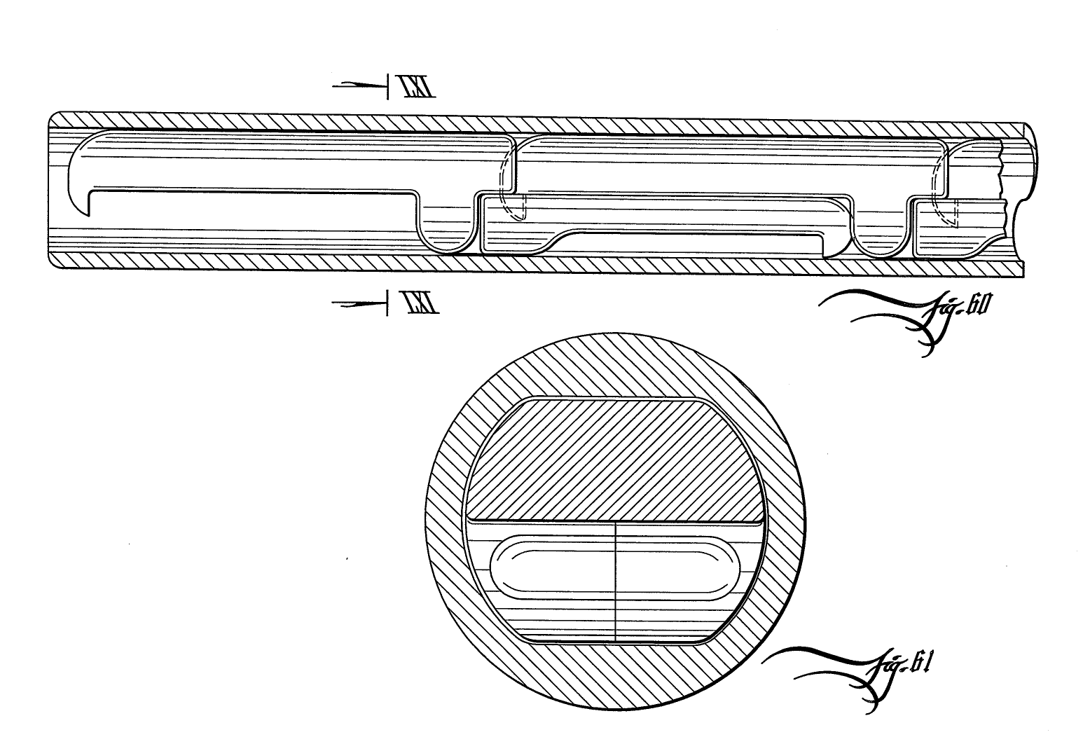

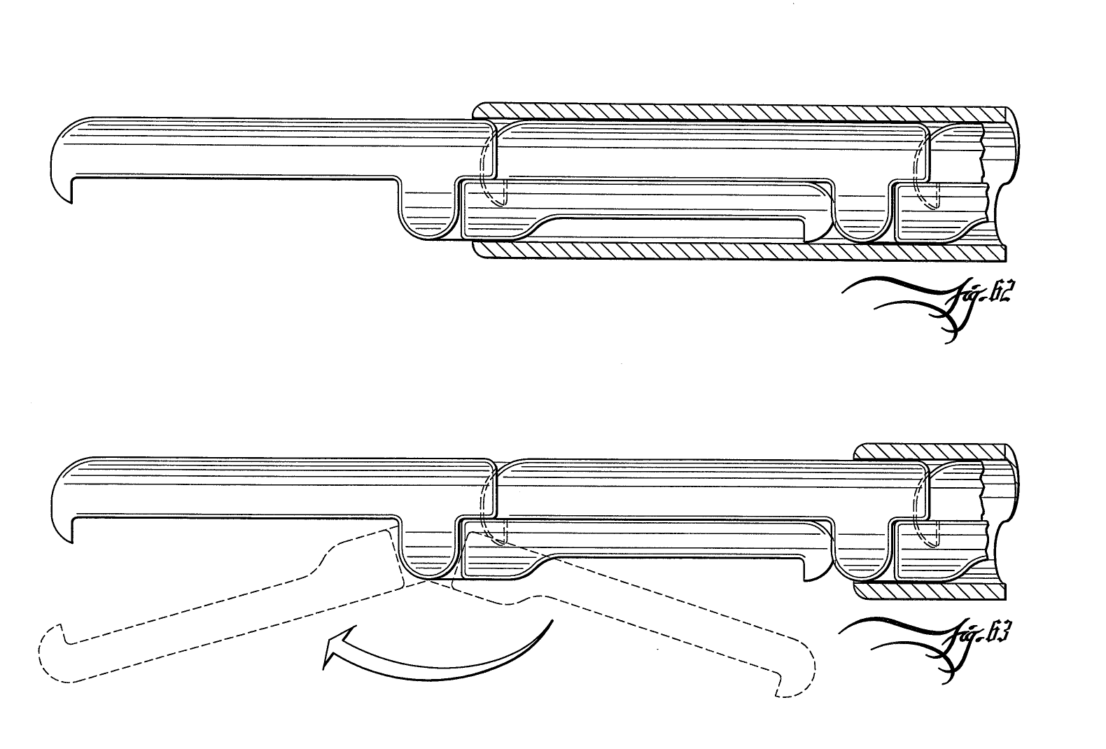

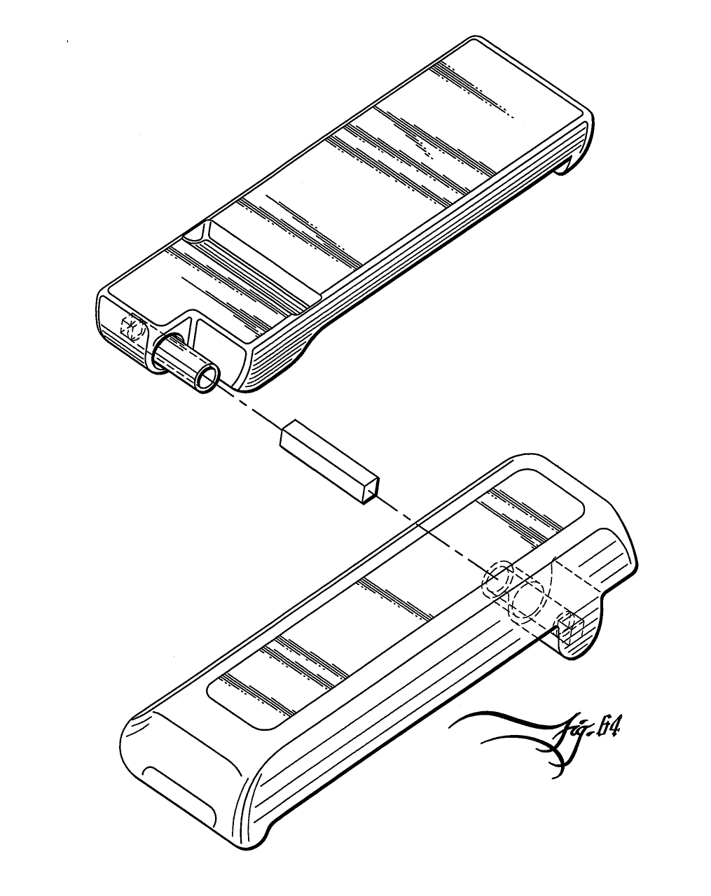

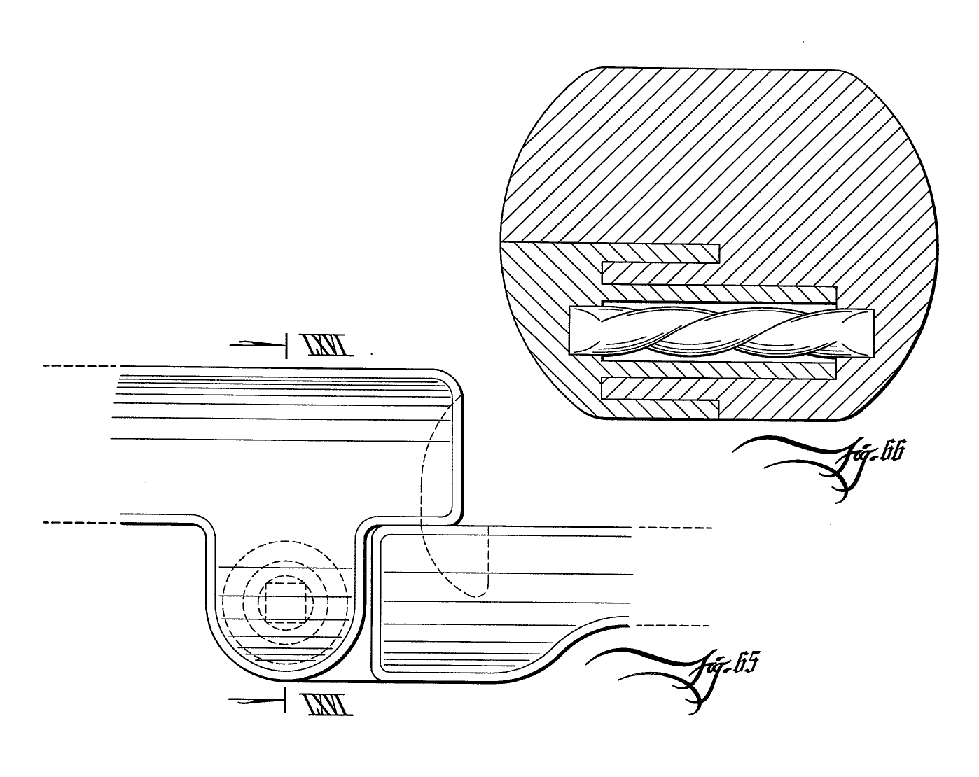

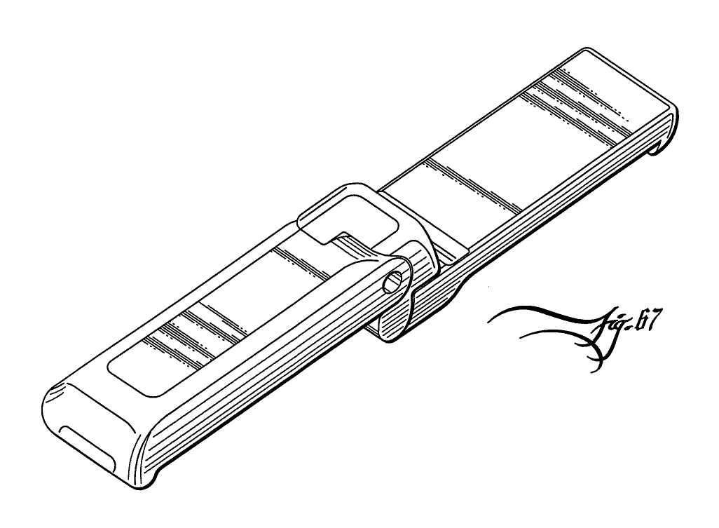

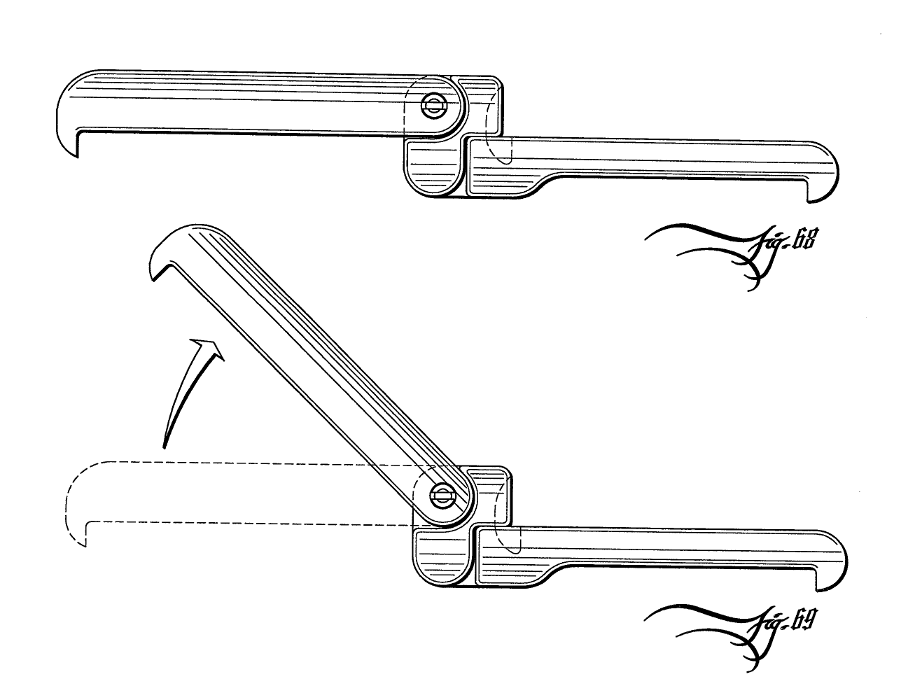

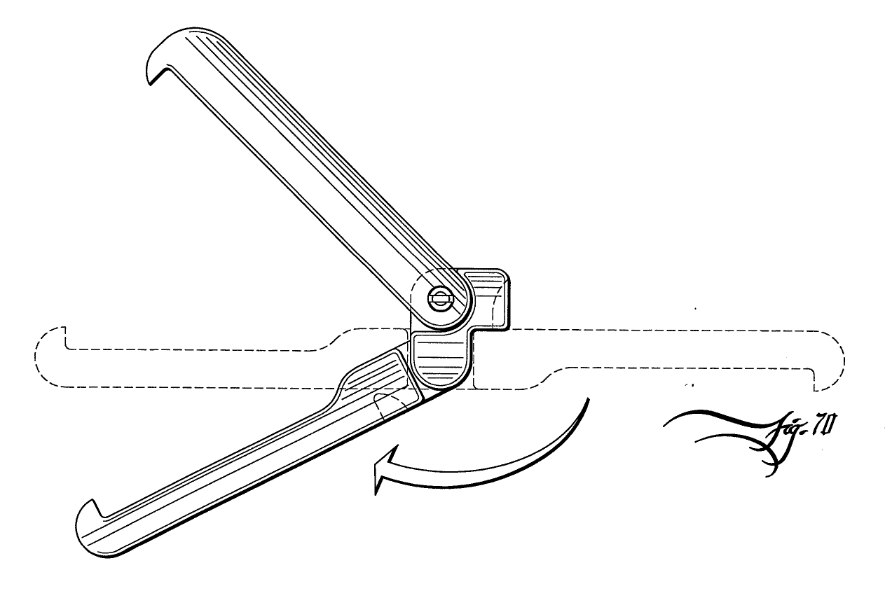

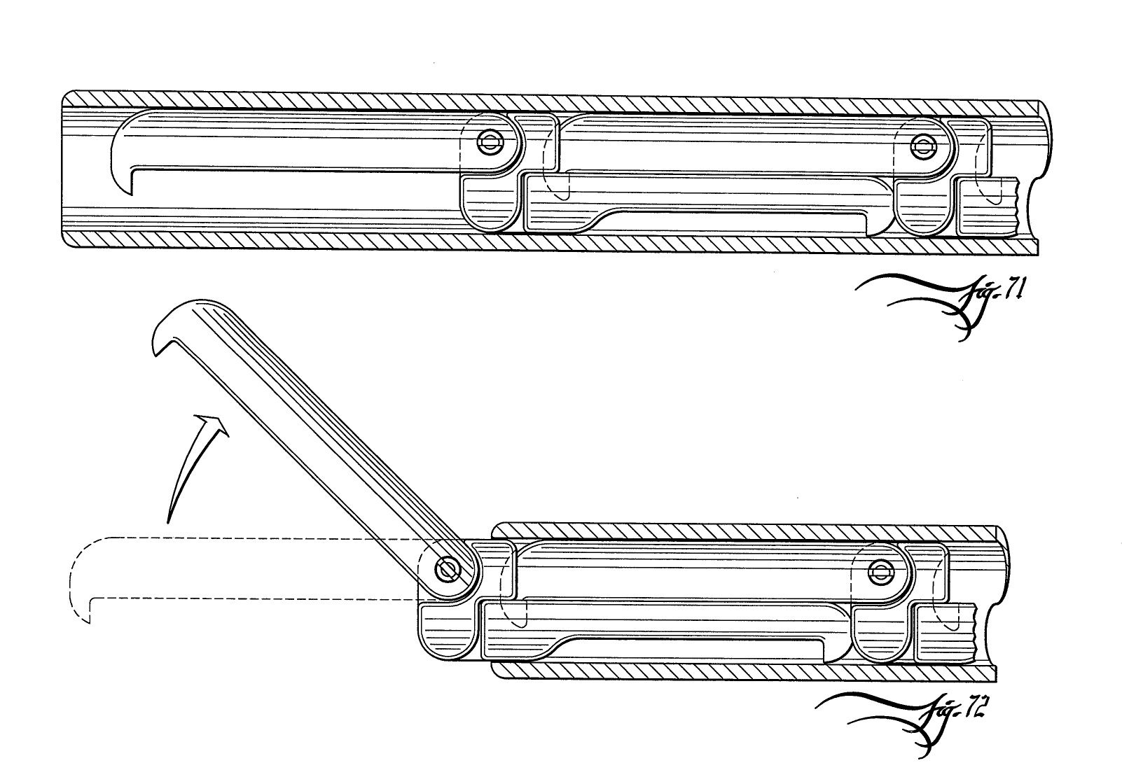

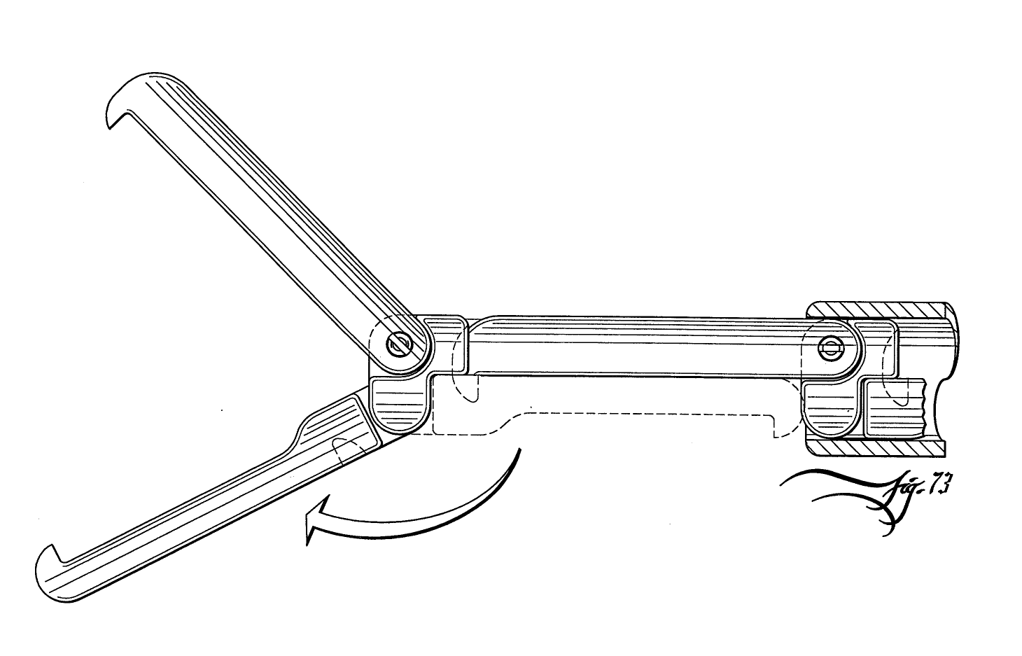

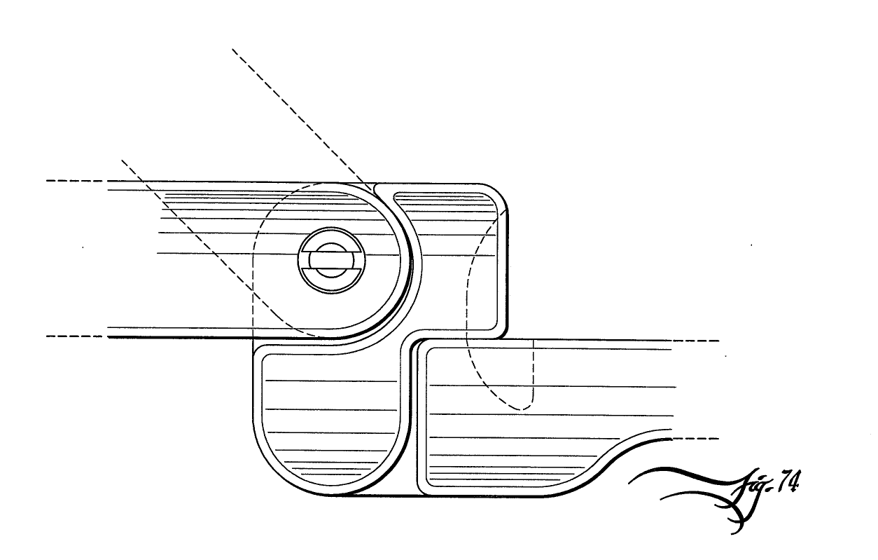

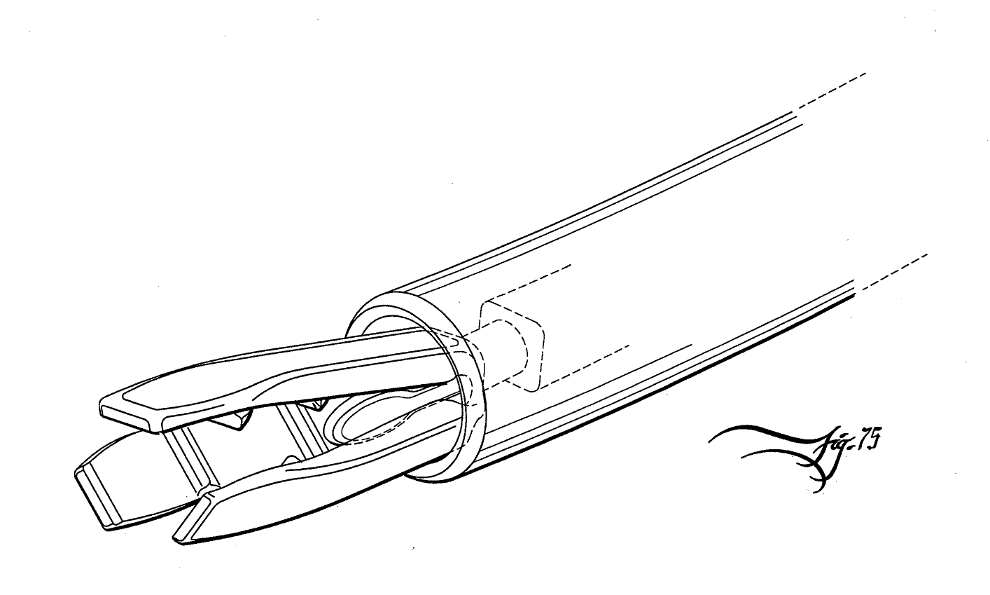

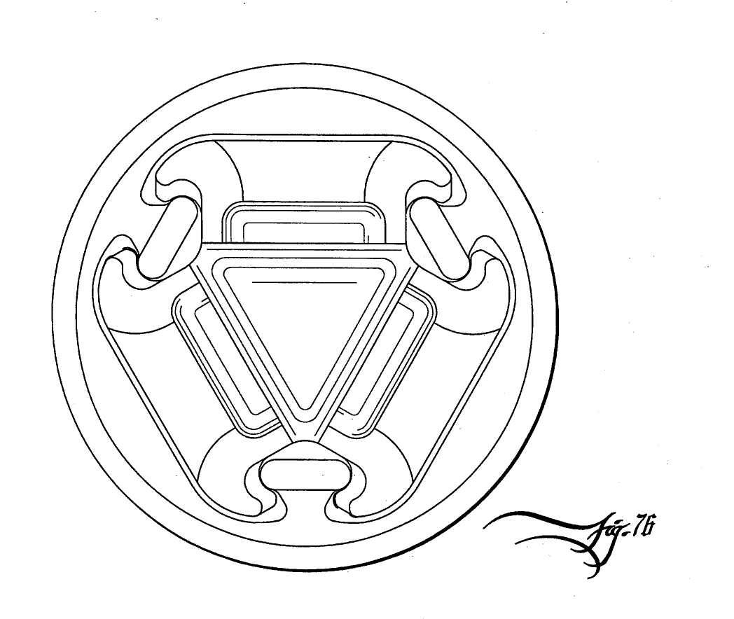

Referring to FIGS. 1-19 and 20-45 show a first and second embodiment respectively wherein the clips have a pair of jaws which face forwardly when loaded in the dispensing apparatus. FIGS. 75 and 76 comparably discloses a third embodiment of a clip when loaded in the dispensing apparatus. FIGS. 46-74 will be dealt with more particularly below.

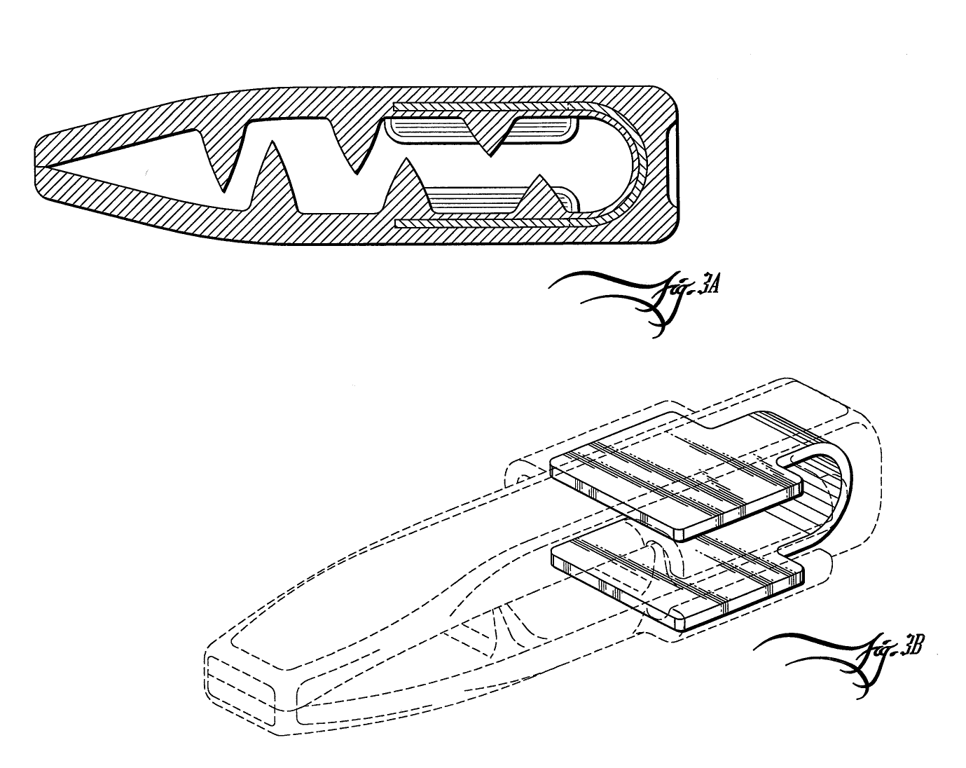

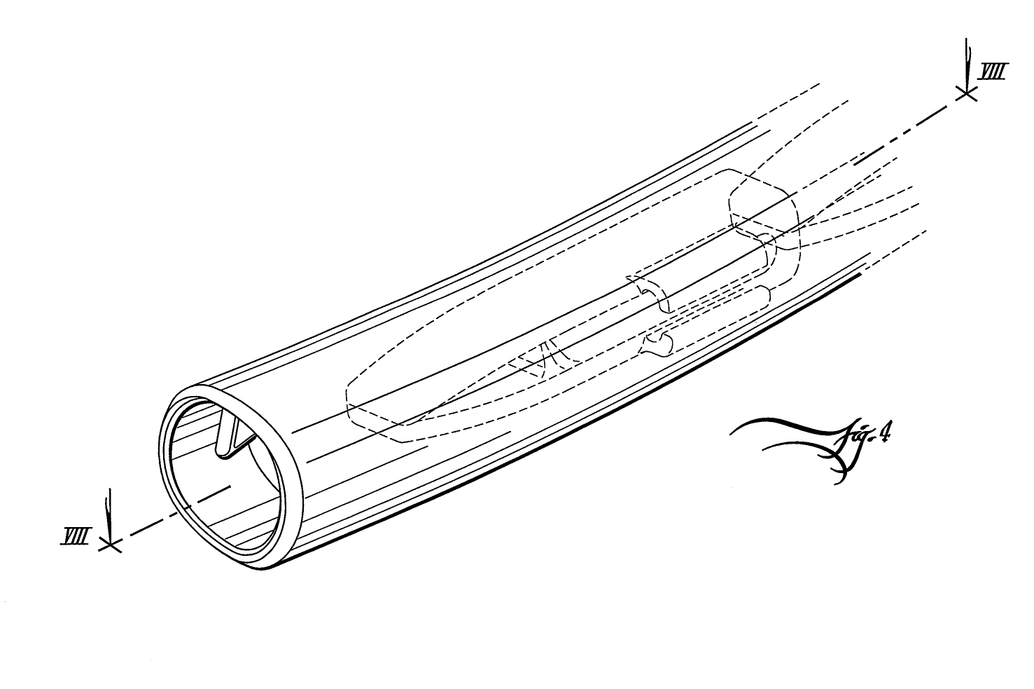

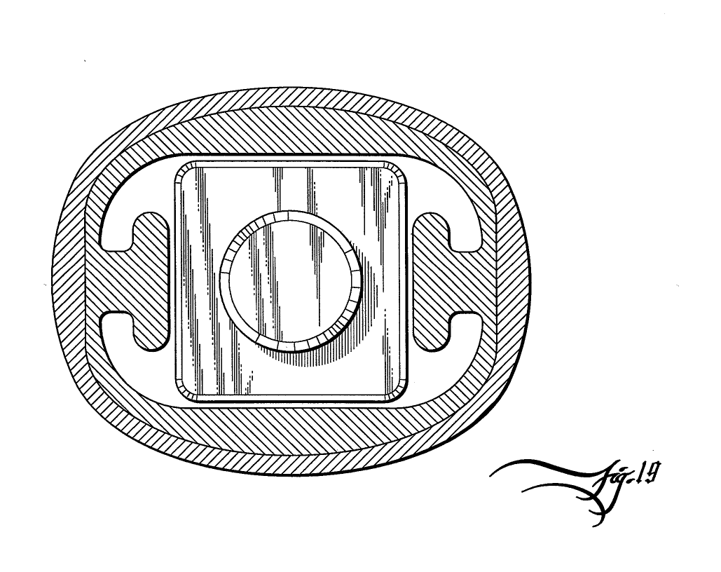

With general reference to FIGS. 1-45, the clip may comprise a composite construction comprising tissue biting jaws permanently biased shut by a clamping spring which has a C-shaped profile from the side (see, eg., FIG. 3B). Alternatively, the clips may comprise a monolithic construction of a single material (see, eg., FIGS. 21-24).

The endoscopic multi-clip dispensing apparatus in accordance with the invention preferably comprises a sleeve inside a sleeve construction, with an inner tubular conduit inserted inside an outer tubular conduit. The outer tubular conduit serves as the frame of reference for the actuation and movement of the other parts of the multi-clip dispensing apparatus. The outer tubular conduit originates in a manual actuator and terminates in a dispensing end.

The inner tubular conduit has a reciprocation action inside the outer tubular conduit, reciprocating between extended and retracted extremes. The inner tubular conduit has a sidewall defining an axial lumen. The series of clips are loaded in the axial lumen of the inner tubular conduit. The series of clips are pushed from behind by an elongated plunger rod serving as a pressure-urging follower. The plunger rod can be indexed through the inner tubular conduit in increments corresponding to the axial length of each clip. That way, the plunger rod pushes forward the line of clips the increment of one clip's length at a time. Thus the plunger rod forces the dispensing of a single clip at a time.

The plunger rod is indexed continually forward at one increment at a time until the last clip is dispensed. Then the plunger rod may be retracted all the way back to an original starting point, such as for (needless to say) the reloading of a fresh line of clips. This can be likened to and without limitation as the way a caulk gun operates on a tube of caulk. The caulk gun plunger rod is ratcheted continually forward in the tube of caulk, forcing the dispensing of caulk out the dispensing end. Once the plunger rod reaches the end, and the tube is depleted, the plunger rod is released to be retracted all the way back to the staring point. The depleted tube is discarded, and a fresh tube is loaded.

As mentioned, the inner tubular conduit has a reciprocation cycle inside the outer tubular conduit. The inner tubular conduit normally rests in its retracted extreme. During an actuation cycle, the inner tubular conduit extends through an extension stroke to an extended extreme. It momentarily holds stationary in the extended extreme as the lead clip is ultimately ejected, after which, the inner tubular conduit returns to rest in its retracted extreme.

The inner tubular conduit has a terminal end which is axially spaced away and inside of the dispensing end of the outer tubular conduit. Hence, resting in the retracted extreme, the terminal end of the inner tubular conduit is gapped away from the dispensing end and inside the core of the outer tubular conduit by a given distance. During the actuation cycle, the terminal end of the inner tubular conduit travels about half that given distance to come closer to the dispensing end of the outer tubular conduit. But, the terminal end of the inner tubular conduit, when reaching the extended extreme, does not travel so far as to actually protrude out of the dispensing end.

Hence the foregoing defines an “ejection station” inside the dispensing end of the outer tubular conduit. That is, the axial length in the core of the outer tubular conduit between its dispensing end, and, the terminal end of the inner tubular conduit in the extended extreme, defines the “ejection station.”

It is in the ejection station that the lead clip is ultimately spread open, ejected, and allowed to form a “bite” of clamping pressure on some target tissue:—which target tissue is targeted by the operator of said endoscopic multi-clip dispensing apparatus in accordance with the invention (eg., a surgeon).

While the inner tubular conduit—speaking strictly of it alone—does not extend out the dispensing end of the outer tubular conduit, the inner tubular conduit does have a pair of arms:—which do. That is. The inner tubular conduit has protruding from its terminal end a pair of arms (at least a pair of arms). That is, if the clips only have a pair of jaws (eg., see, FIGS. 1-45) then the inner tubular conduit will have just a pair of arms. However, if the clips have three jaws (eg., see, FIGS. 75 and 76), then the inner tubular conduit will have three arms. And so on, the number of arms roughly corresponding to the number of jaws.

The arms project from base ends anchored in the inner tubular conduit proximate the terminal end thereof, to free ends. When the inner tubular conduit is resting in its retracted position, the free ends of the arms lie just inside the dispensing end of the outer tubular conduit. When the inner tubular conduit is extended to its extended extreme, the free ends of the arms do indeed project outside of the dispensing end of the outer tubular conduit.

The free ends of the arms are formed in wedge-shaped flared ends, which also are flexed in toward each other, each at the end of a respective arm. Preferably the arms are produced out not only a resilient material but a durable material too, one which resists erosion after the scraping passage thereover of numerous clips. It is preferred without limitation that the arms are produced of spring steel.

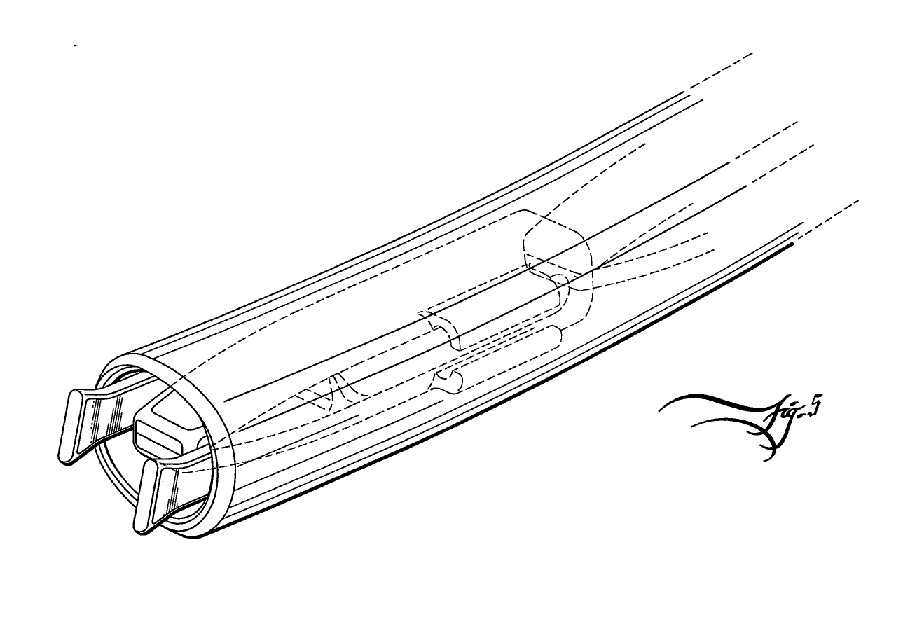

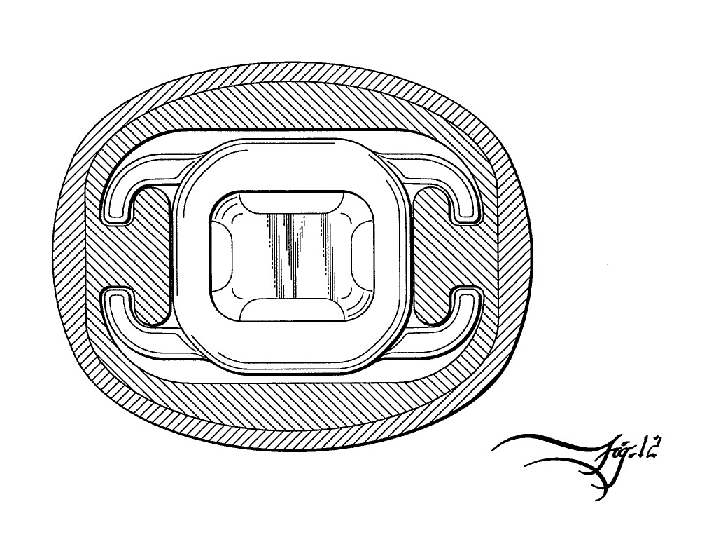

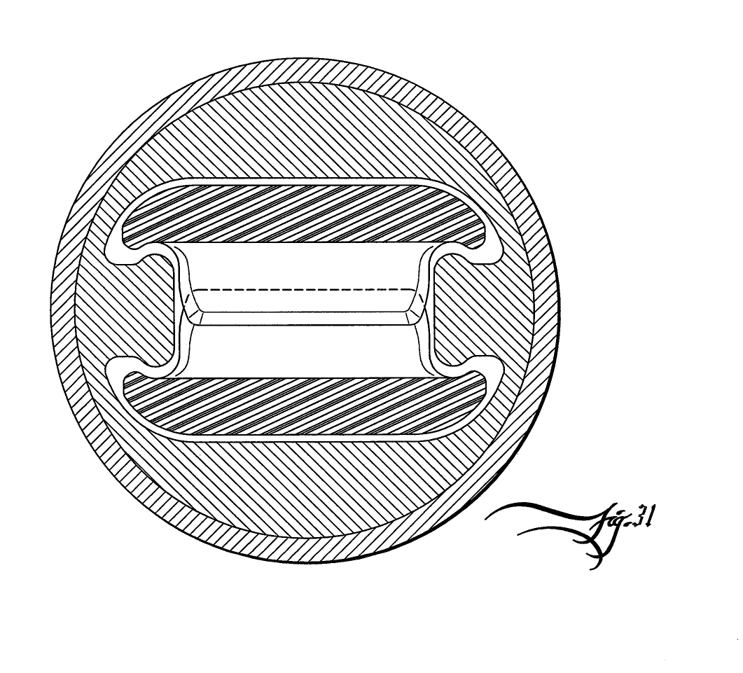

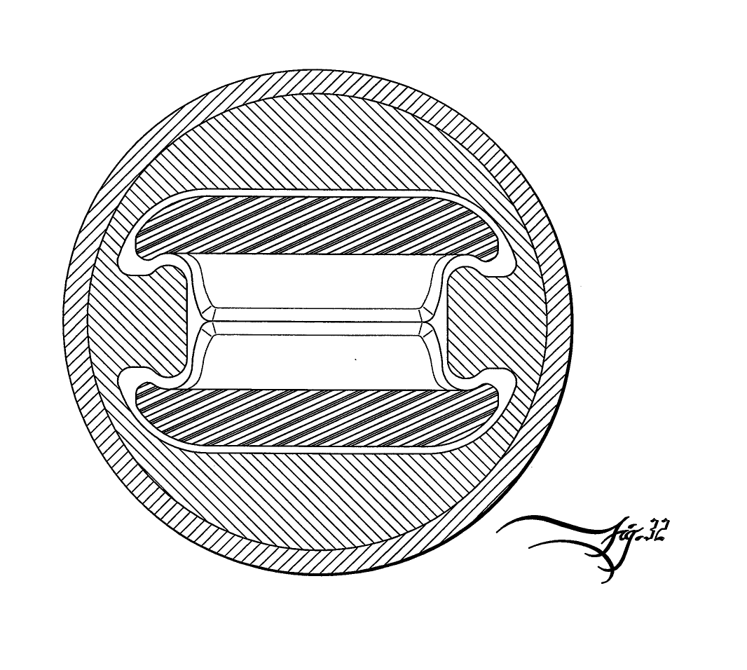

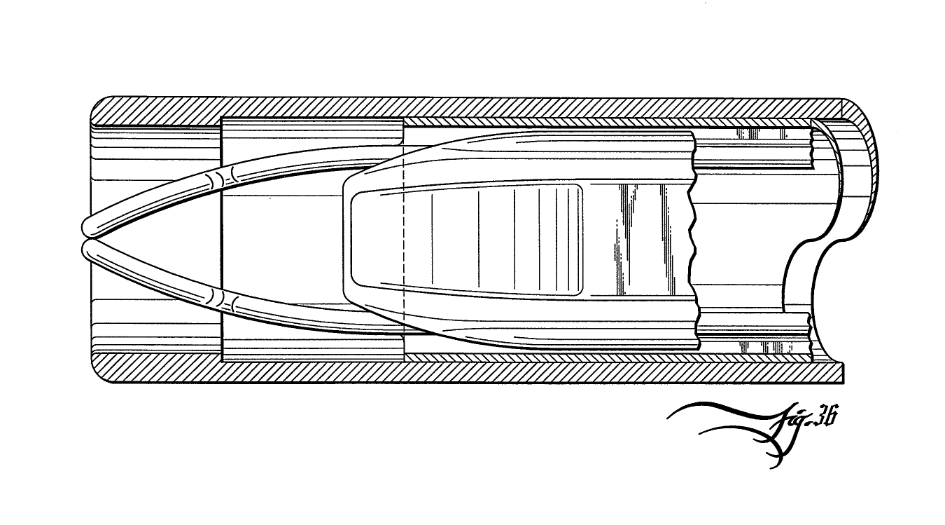

The drawings figures provide several sequences of views showing how one clip is ultimately dispensed. See, eg., FIGS. 4-7, and/or FIGS. 13-17, and/or FIGS. 34-35, and/or FIGS. 36-39, and/or FIGS. 40-45, and so on.

Therefore, any of the foregoing sequences of views show a progression comprising the lead clip of a series of clips in accordance with the invention being dispensed out the dispensing end of the endoscopic multi-clip dispensing apparatus in accordance with the invention.

The dispensing apparatus is shown in section in the various sequences identified above, and from various perspectives, or from vertical and horizontal cutting planes through the longitudinal axis thereof. The series of clips are typically shown in a head to tail line up, which each sequence beginning with the lead clip in position for dispensing.

The sequences show the progression of changes the lead experiences through the process of being dispensed. The lead clip is being pushed from behind by a considerable thrusting force on the line of clips in the dispenser's inner tubular conduit. As do all of the clips, the lead clip has upper and lower jaws giving the clip an overall C-shape. Both the upper and lower jaws have turned-in flanges or turned-in channels defining eaves or tracks that ride along the edges up the wedge-shaped flared ends of the arms. With the eaves or tracks of the C-shaped clip being spread open by scraping transit over the wedge-shaped flared ends of the arms, the lead clip's tissue biting jaws open, and, open wide.

In order for the jaws to open that wide, the wedge-shaped flared ends of the arms have to reciprocate through a cycle of extension and retraction strokes. The flared ends extend outside of the dispensing end of the outer tubular conduit in order to give the tail end of the clip sufficient clearance to flex open wide. Otherwise, if confined in the ejection station, the lead clip would not be able to open as wide. As the eaves or tracks of the lead clip ultimately pass by the flared ends of the extended arms, the jaws of the lead clip are free to snap shut. At the same time, the lead clip is released from any further contact with the dispenser or line of trailing clips. The lead clip is free to bite firmly on any tissue which the operator has aimed the lead clip at (eg., an example operator is a surgeon). In the last stage of the ejection of the lead clip, the line of trailing clips from behind move forward until what becomes the succeeding new lead clip is trapped in a ready-to-go, next-to-be ejected position by the retracting arms and inward flexing flared ends of inner tubular conduit. That is, the wedge-shaped flared-ends retract by the retraction stroke of the inner tubular conduit and form somewhat of an clapped together embrace with each other by the resiliency in the arms. This clapping embrace of the flared ends can be likened to, and without limitation, a single clap of a person's hand.

To turn now to FIGS. 46-74, these views show several embodiments of clips and dispensing apparatus arranged wherein the jaws open to about 180° apart when loaded in the tubular conduit of the dispensing apparatus. In such an arrangement, one jaw is the lead jaw and the other jaw is the trailing jaw. The leading jaw has a leading hook-end and the trailing jaw has a trailing hook-end. During the progression of dispensing the leading clip out of the dispenser end, the leading clips leading end extends out first. Preferably the leading hook-end of the leading jaw is latched onto some target tissue so the that clip does not wastefully close on itself without clamping any tissue. Then when the trailing jaw clears the dispensing end of the dispensing apparatus, the trailing jaw snaps shut toward the leading jaw, and thereby forms the intended clamping action.

The dispensing apparatus only needs a single tubular conduit, and not a sleeve-inside-a sleeve configuration as before. However, the dispensing apparatus still needs a plunger rod incrementally forcing each clip in series out of the dispensing end, one clip at a time. Preferably any give clip ahead of another in the line of clips is pushed forward by the leading end of the trailing clip bearing against the hinge of the leading clip.

It is further preferred if the core of the tubular conduit had a metering device such as and without limitation a miniature turn-stile or the like proximate the dispensing end. The metering device would preferably meter the dispensing of the clips to one clip at a time. That is, as soon as the current-lead clip is ejected, the metering device holds back or offers resistance to the next clip. However, the means holding back the next clip or offering the resistance can be overcome after a moment's wait to thereby allow the operator (eg., surgeon) to eject the next clip.

The invention having been disclosed in connection with the foregoing variations and examples, additional variations will now be apparent to persons skilled in the art. The invention is not intended to be limited to the variations specifically mentioned, and accordingly reference should be made to the appended claims rather than the foregoing discussion of preferred examples, to assess the scope of the invention in which exclusive rights are claimed.

I claim:

1. A combination endoscopic multi-clip dispensing apparatus and a plurality of clips therefor, comprising:
an elongated inner tubular conduit sleeved inside an elongated outer tubular conduit, wherein the inner tubular conduit reciprocates between extension and retraction strokes;
a plurality of clips, each having a C-shape characterized by a pair of elongated jaws mated together at a hinge end, wherein the jaws have tip ends forming the head end for the clip and hinge end forms the tail end for the clip when said plurality of clips are loaded into core of the inner tubular conduit in head to tail fashion;
a plunger rod for pushing the line of clips through the core of the inner tubular conduit at an incrementally indexed or metered rate of one clip at a time; and
a pair of wedge-shaped flared structures proximate the dispensing end of one of the outer tubular conduit or inner tubular conduit for prying open the jaws of the lead clip during the progression of the lead clip being dispensed whereby, after the lead clip passes clear of the flared structures, the jaws thereof are free to snap shut and ‘bite’ into a target anatomical structure.
